(19) United States
(12) Patent Application Publication
(10) Pub. No.: US 2013/0209402 A1
(43) Pub. Date: Aug. 15, 2013

(54) HUMAN PAPILLOMAVIRUS E7 ANTIGEN COMPOSITIONS AND USES THEREOF

(71) Applicants: John R. Webb, North Saanich (CA); Darin Arne Wick, Victoria (CA)

(72) Inventors: John R. Webb, North Saanich (CA); Darin Arne Wick, Victoria (CA)

(73) Assignee: BRITISH COLUMBIA CANCER AGENCY BRANCH, VANCOOUVER BC (CA)

(21) Appl. No.: 13/810,352

(22) PCT Filed: Jul. 15, 2011
(86) PCT No.: PCT/CA11/00823

§ 371 Date: Apr. 29, 2013

Related U.S. Application Data
(60) Provisional application No. 61/364,750, filed on Jul. 15, 2010.

(51) Int. Cl. C07K 2/00

(52) U.S. Cl. CPC C07K 2/00
USPC 424/85.7; 530/300; 536/23.72; 424/186.1; 424/173.1; 530/328; 530/326; 435/5

(57) ABSTRACT

The present invention relates to human papillomavirus E7 antigen compounds and compositions for treating human papillomavirus infection and associated conditions. The invention provides, in part, polypeptide and nucleic acid molecules including sequences substantially identical to the sequences of two or more human papillomavirus (HPV) E7 antigens, where the E7 antigens are selected from at least two different HPV strains, and methods of using the same.

### FIELD OF INVENTION

The present invention relates to compounds and compositions for treating human papillomavirus infection and associated conditions. More specifically, the present invention relates to human papillomavirus E7 antigen compounds and compositions for treating human papillomavirus infection and associated conditions.

### BACKGROUND OF THE INVENTION

Human papillomaviruses (HPV) are a group of more than 100 related, but genetically distinct, ‘types’ of virus, which can be broadly classified as low-risk and high-risk. Low risk HPV types are associated with common warts (or papillomas) that are generally benign and non-life threatening. In contrast, persistent infection with high-risk types of HPV is associated with pre-cancerous cervical dysplasia and cervical cancer (1,2). High risk HPV types are also associated with cancers of the anus, vulva, vagina, and penis (3) as well as certain subsets of head and neck cancers (4-6) and breast cancer (57).

High risk HPV types that cause cervical cancer are sexually transmitted and are highly prevalent in the normal, healthy population. It is estimated that a majority of people become infected shortly after becoming sexually active (7, 59). The vast majority of high risk HPV infections are thought to be self-limiting, with minimal associated pathology, however infection with high risk HPV may develop into malignancy (8).

While HPV16 and HPV18 are the most prevalent of the high risk HPV types, there are many additional high risk types of HPV. A recent comparative analysis of 1,918 cervical cancer patients and 1,928 healthy control women revealed that the most common HPV types in cancer patients (in descending order of frequency) were HPV types 16, 18, 45, 31, 33, 52, 58 and 35 (11). Likewise, a global analysis of the prevalence of HPV in cervical cancer revealed that HPV16 was present in 50% of cases, HPV18 in 14%, HPV45 in 8%, and HPV31 in 5%, with other members of the thirteen “high risk” types making up the remaining cases (12).

Due to their prevalence, HPV 16 and HPV 18 have been the focus of a broad prophylactic immunization campaign aimed at preventing initial infection by HPV (9, 10) and currently approved prophylactic vaccines target the high risk strains HPV16 and HPV18 (as well as the low risk strains HPV6 and HPV 11) in anticipation that prophylactic vaccination will diminish or prevent the occurrence of HPV-associated malignancy later in life. It has also been suggested that HPV 16 and HPV 18 prophylactic vaccines may confer partial cross-protection against other high risk types of HPV (13-15).

Prophylactic HPV vaccines currently in clinical use are comprised of recombinant viral capsid glycoprotein (L1) that spontaneously forms synthetic virus-like particles (VLP) (60, 61). Immunization with VLP-based, prophylactic vaccines elicits a strong, neutralizing antibody response against the L1 protein, which prevents viral infection from becoming established.

Prophylactic vaccines however appear to have minimal impact on established infection (16). For example, prophylactic cervical cancer vaccines are ineffective for those individuals who have already been exposed to HPV, since once the virus gains entry into the cell, it is protected from the neutralizing effects of extracellular antibody, allowing viral replication (and latent infection) to proceed unimpeded. Infection with high risk HPV may result in integration of the viral episome into host DNA, often resulting in deletion of several early (E2, E4 and E5) and late (L1 and L2) genes, leaving the HPV proteins, E6 and E7, as the only viral proteins that continue to be expressed in the infected cell (23, 24, 59). In this situation, vaccine-induced immunity against the L1 capsid protein is ineffective for therapy.

In addition, because of the high prevalence of HPV infection in today's adult population combined with the slow progress of cervical carcinogenesis, it is anticipated that it will take 20 years, or more, until mass implementation of a prophylactic vaccine will have an impact on the incidence of cervical cancer. Also, prophylactic vaccines have met with resistance in some instances and the rate of vaccination is variable (17). In some areas, vaccine cost remains an issue (18).

Therapeutic HPV vaccines, designed to eradicate pre-existing lesions by generating cellular immunity against HPV-infected cells that express viral proteins, have been explored as an alternative for treatment of HPV-associated cancer (for review see 19, 20, 62-65) and many of these approaches have been aimed at the development of vaccines that elicit a robust CD8 T cell response since many vaccines currently approved are generally poor at eliciting CD8 immunity (66, 67). HPV E7 therapeutic vaccination approaches have included peptide immunization (26, 28-30), DNA immunization (31-33, 68), immunization with recombinant, E7-expressing Vaccinia virus (25, 34), adenovirus (35-37), Salmonella typhimurium (38, 39) or Listeria monocytogenes (40, 41), E7-pulsed dendritic cells (42-45) or E7-containing virus-like particles (VLP) (46-49) and a number of these therapeutic vaccine strategies have advanced to early clinical trials.

Many of these therapeutic vaccine strategies are often logistically cumbersome and the responses elicited rarely reach the level of CD8 T cell expansion that is seen during the acute phase of an authentic anti-viral immune response (69). Strategies for enhancing CD8 immunity via booster vaccination, such as various types of heterologous prime-boost regimens, including DNA-peptide, DNA-virus or two distinct viral vehicles for prime-boost, have been used for eliciting CD8 immunity (70-72). However, these methods can be difficult to translate to the clinic and there is poor consensus regarding which methodologies are optimal.

HPV vaccines have been described in a number of publications including PCT publications WO2005/089164 (published Sep. 29, 2005), WO2007/121894 (published Nov. 1, 2007), WO2007/121895 (published Nov. 1, 2007), WO2008/049329 (published May 2, 2008), and WO2008/145745 (published Dec. 4, 2008).

The TC-1 model tumor system, originally derived from mouse primary lung epithelial cells that were transformed with HPV 16 E6 and E7 oncogenes, which are required for transformation and immortalization of infected cells and maintenance of the cells in a transformed state (21, 22), along with activated human c-Ha-ras (25), has become widely adopted as a test system for HPV therapeutic vaccines. Implantation of TC-1 tumor cells into immunocompetent C57B1/6 mice results in the formation of rapidly progressing tumors at the site of inoculation. However, specific cellular immunity against the HPV 16 E7 protein can confer protection against TC-1 tumor outgrowth. For example, CD8+ T cells specific for the H-2 Db-restricted epitope (E749-57; RAHYNIVTF) of E7 have been reported to be capable of lysing E7-expressing tumor cells and causing regression of established TC-1 tumors (26, 27).

Immunization with whole exogenous protein has been suggested to be an inefficient means of eliciting MHC class I-restricted CD8+ T cell responses (50). However, immunization with recombinant (51) or synthetic (52) full length E7 protein has been reported to elicit CD8+ T cell immunity when delivered in combination with either QuilA or CpG-containing oligonucleotides, respectively. Likewise, recombinant proteins comprised of fusions between immunogenic heat shock proteins (HSP) and selected target antigens are also reported to elicit CD8+ immunity against the target antigen. It has been suggested that immunization with whole exogenous protein plus TLR3 or TLR9 agonists facilitates the process of cross-priming and promotes the development of antigen-specific CD8+ T cell responses (55, 56).

Currently, cervical dysplasia and early stage cervical cancer are most commonly treated using a surgical procedure known as LEEP (Loop electrosurgical excision procedure) in which abnormal tissue is removed using a thin wire loop charged with an electrocurrent. More advanced stages of cervical cancer are treated by surgery (partial or radical hysterectomy) combined with chemotherapy and or radiation therapy.

### SUMMARY OF THE INVENTION

The invention provides, in part, human papillomavirus E7 antigen compounds and compositions. The compounds and compositions may be useful for treating or diagnosing human papillomavirus infection and associated conditions.

In one aspect, the invention provides a polypeptide including an amino acid sequence substantially identical to the amino acid sequence of two or more human papillomavirus (HPV) E7 antigens, where the E7 antigens are selected from at least two different HPV strains.

In alternative embodiments, the different HPV strains may be high risk strains, such as HPV16, HPV18, HPV31, HPV33, HPV35, HPV39, HPV45, HPV51, HPV52, HPV56, HPV58, HPV59, HPV68, HPV73, or HPV82. In alternative embodiments, the E7 antigens may be selected from five different HPV strains, such as HPV16, HPV18, HPV31, HPV45, and HPV52.

In alternative embodiments, the polypeptide may include two or more of the amino acid sequences set forth in SEQ ID NOs: 1 to 15, or the amino acid sequences set forth in SEQ ID NOs: 1 to 5, such as the amino acid sequence set forth in SEQ ID NO: 16 or 17.

In alternative embodiments, the polypeptide may be encoded by a nucleotide sequence comprising two or more of the nucleotide sequences set forth in SEQ ID NOs: 18 to 32.

In alternative embodiments, the polypeptide may be encoded by a nucleotide sequence comprising two or more of the nucleotide sequences set forth in SEQ ID NOs: 18 to 22, such as SEQ ID NOs: 33 or 34.

In alternative embodiments, the E7 antigens may be capable of inducing an immune response to the two different HPV strains.

In other aspects, the invention provides a nucleic acid molecule including a sequence substantially identical to the nucleotide sequences of two or more human papillomavirus (HPV) E7 antigens, where the E7 antigens are selected from at least two different HPV strains.

In alternative embodiments, the different HPV strains may be high risk strains, such as HPV16, HPV18, HPV31, HPV33, HPV35, HPV39, HPV45, HPV51, HPV52, HPV56, HPV58, HPV59, HPV68, HPV73, or HPV82.

In alternative embodiments, the E7 antigens may be selected from five different HPV strains, such as HPV16, HPV18, HPV31, HPV45, and HPV52.

In alternative embodiments, the nucleic acid molecule may include two or more of the nucleic acid sequences set forth in SEQ ID NOs: 18 to 32.

In alternative embodiments, the nucleic acid molecule may include the nucleic acid sequences set forth in SEQ ID NOs: 18 to 22, such as SEQ ID NOs: 33 or 34.

In alternative aspects, the invention provides a nucleic acid molecule encoding a HPV E7 polypeptide.

In alternative aspects, the invention provides an expression vector including a nucleic acid sequence as described herein operably linked to a sequence that allows for expression of the nucleic acid sequence in a host cell.

In alternative aspects, the invention provides a host cell comprising a nucleic acid molecule or expression vector as described herein.

In alternative aspects, the invention provides a composition including a polypeptide, nucleic acid molecule, expression vector or host cell as described herein. The composition may include a carrier and/or an adjuvant. The adjuvant may be a Toll-like receptor (TLR) agonist such as a TLR3 agonist (e.g., poly(I:C)) or a TLR9 agonist (e.g., a CpG containing oligonucleotide). Alternatively or additionally, the adjuvant may be an interferon-alpha, an agonist of the 4-1 BB receptor, an agonist of the CD40 receptor, or an anti-CD40 antibody.

In alternative aspects, the invention provides a method of stimulating an immune response in a subject in need thereof by administering a polypeptide, nucleic acid molecule, expression vector or host cell as described herein, to the subject. In alternative aspects, the invention provides a method of treating or preventing a condition associated with HPV infection in a subject in need thereof, by administering a polypeptide, nucleic acid molecule, expression vector or host cell as described herein, to the subject. In alternative aspects, the invention provides a method of treating a HPV infection in a subject in need thereof, by administering a polypeptide, nucleic acid molecule, expression vector or host cell as described herein, to the subject.

In alternative aspects, the invention provides a use of a polypeptide, nucleic acid molecule, expression vector or host cell as described herein, for stimulating an immune response in a subject in need thereof. In alternative aspects, the invention provides a use of a polypeptide, nucleic acid molecule, expression vector or host cell as described herein, for treating or preventing a condition associated with HPV infection in a subject in need thereof. In alternative aspects, the invention provides a use of a polypeptide, nucleic acid molecule, expression vector or host cell as described herein, for treating a HPV infection in a subject in need thereof.

The condition associated with HPV infection may be one or more of a cancer of the breast, cervix, anus, vulva, vagina, penis, head and neck, and lung, or pre-malignant lesion thereof, or may be a pre-cancerous cervical epithelial neoplasia (CIN I through CIN III) or a cervical cancer.

In alternative embodiments, the HPV infection may be by a high risk HPV type, such as HPV16, HPV18, HPV31, HPV33, HPV35, HPV39, HPV45, HPV51, HPV52, HPV56, HPV58, HPV59, HPV68, HPV73, or HPV82. In alternative embodiments, the methods or uses may further include administering an adjuvant such as a Toll-like receptor (TLR) agonist (e.g., TLR3 agonist like poly(I:C) or a TLR9 agonist like a CpG containing oligonucleotide). Alternatively or additionally, the adjuvant may include an interferon-alpha, an agonist of the 4-1BB receptor, an agonist of the CD40 receptor, or an anti-CD40 antibody. The administering may include administration of multiple doses over a time frame of less than 14 days, or may include administration of multiple doses over one to four days, and/or may include administration of multiple daily doses.

In alternative aspects, the invention provides a peptide consisting essentially of one or more of the sequences TSNYNIVTF (SEQ ID NO: 35), AEPDTSNYNIVTFCC (SEQ ID NO: 36) or TSNYNIVTFCCQCKS (SEQ ID NO: 37). In alternative aspects, the invention provides a method of diagnosing a HPV31 infection comprising contacting a sample with a peptide consisting essentially of one or more of the sequences TSNYNIVTF (SEQ ID NO: 35), AEPDTSNYNIVTFCC (SEQ ID NO: 36) or TSNYNIVTFCCQCKS (SEQ ID NO: 37). In alternative aspects, the invention provides a method of determining the response of a subject to a HPV31 infection contacting a sample with a peptide consisting essentially of one or more of the sequences TSNYNIVTF (SEQ ID NO: 35), AEPDTSNYNIVTFCC (SEQ ID NO: 36) or TSNYNIVTFCCQCKS (SEQ ID NO: 37).

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A-B are schematic diagrams showing the structural organization of the Pentarix protein (A) without an affinity tag or (B) with a cleavable 6× affinity tag and a thrombin cleavage site.

FIG. 1C shows the preparation and purification of Pentarix protein with a cleavable 6× affinity tag. The Figure shows expression of recombinant Pentarix protein in E coli and an example of a typical purification using nickel affinity purification. Total protein contained within a lysate of IPTG-induced E. coli before and after passage over a HisTrap column (GE Healthcare) and protein eluted from the column (Fr#1-12) in the presence of increasing concentrations of imidazole were detected by Coommassie Blue staining (left panel). Identity of purified Pentarix protein was confirmed by Western blot analysis of lysates from un-induced and induced cultures as well as purified Pentarix and HPV 16 E7 proteins using anti-6×His tag antibody or anti-HPV 16E7 antibody.

FIG. 2A is a graph showing OVA₂₅₇₋₂₆₄-specific CD8₊ T cell responses elicited in response to immunization with whole exogenous OVA protein plus the TLR3 agonists poly(I:C) or polyIC/LC. Naïve C57B1/6 mice (2 mice per condition) were immunized with whole OVA protein (500 μg) plus or minus poly(I:C) (10 μg) or polyIC/LC (10 μg/ml). Seven days post-immunization mice were euthanized and the number of OVA₂₅₇₋₂₆₄ (SIINFEKL)-specific CD8+ T cells in bulk splenocytes of immunized mice were quantitated by IFN-γ ELISPOT. Results are reported as the number of IFN-γ spot-forming cells per 1×10⁶ splenocytes after stimulation with media only, SIINFEKL peptide (10 μg/ml) (SEQ ID NO: 39) or irrelevant H2 Db-binding peptide (10 μg/ml).

FIG. 2B is a graph showing that OVA₂₅₇₋₂₆₄-specific CD8 T cell responses are elicited in dose-dependent manner after immunization with whole exogenous OVA protein plus the TLR3 agonist poly(I:C).

FIGS. 3A-E show OVA₂₅₇₋₂₆₄-specific CD8+ T cell or HPV16 E7 responses elicited in response to long or short interval (cluster) homologous prime-boost immunization. A, Naïve C57B1/6 mice (2 mice per condition) were immunized with whole OVA protein (100 μg) plus poly(I:C) (10 μg) at day −7, day −21 or day −7 and day −21 and were euthanized at day 0. The number of OVA₂₅₇₋₂₆₄-specific CD8+ T cells in bulk splenocytes of immunized mice were quantitated by IFN-γ ELISPOT. B, Naïve C57B1/6 mice (3 mice per condition) were immunized with the indicated number of sequential daily doses of whole, soluble OVA protein (100 μg) admixed with poly(I:C) (10 μg). One additional group of mice received a single immunization that was equivalent to four times the normal daily dose (i.e. 400 μg of OVA protein plus 40 μg of poly(I:C)). Seven days after the first immunization mice were euthanized and bulk splenocyte preparations were assessed by IFN-γ ELISPOT to quantitate the number of OVA₂₅₇₋₂₆₄-specific CD8+ T cells. Results in A and B are reported as the number of IFN-γ spot-forming cells per 1×10⁶ splenocytes after stimulation with media only or SIINFEKL peptide (10 μg/ml). C, Naïve C57B1/6 mice were immunized with one dose or four consecutive daily doses of whole, soluble OVA protein (100 μg) plus poly(I:C) (10 μg) as indicated. D, Mice in panel c that received four consecutive daily doses of OVA protein plus poly(I:C) were reimmunized with another four consecutive daily doses of the same, starting at day 47 after imitation of the first round of immunization. Peripheral blood was obtained from the saphenous vein of individual mice that were serially bled on the indicated days post-immunization. RBC in peripheral blood were lysed and lymphocytes were stained with FITC-conjugated anti-CD8 and PE-conjugated H-2 Kb/OVA₂₅₇₋₂₆₄ tetramer and analyzed by flow cytometry. Events shown in C and D are gated on CD8+ lymphocytes and are from a representative single animal to allow precise monitoring of the evolution of the antigen-specific T cell responses within a given animal over time. E, Naïve C57B1/6 mice were immunized with one dose (left panel), four consecutive daily doses of whole, soluble HPV16 E7 protein (100 ug) plus poly(I:C) (10 ug), or four consecutive daily doses of whole, soluble HPV16 E7 protein (100 ug) only. Seven days post-immunization, peripheral blood was obtained from immunized mice. Lymphocytes were stained with FITC-conjugated anti-CD8 and PE-conjugated D^{b}/E7₄₉₋₅₇ tetramer and analyzed by flow cytometry. Events shown are gated on CD8⁺ lymphocytes.

FIG. 4 is a series of graphs showing sequential daily immunization with whole, soluble protein plus the TLR3 agonist poly(I:C) induces regression of large, established tumors. C57B1/6 mice (3 mice per cohort) were implanted with OVA-expressing EG7 tumors cells (1×10⁵) on day 0 and were left untreated (upper left), or were treated with 1 dose of poly(I:C) (10 μg) (upper right), 1 dose of whole, soluble OVA protein (100 μg) plus poly(I:C) (10 μg) (lower left) or four sequential daily doses of whole, soluble OVA protein (100 μg) plus poly(I:C) (10 μg)) (lower right). Time of treatment for each group is indicated by the arrowhead(s). Average tumor volume at time of treatment for each group was 224 mm³ (poly(I:C) only), 194 mm³ (1 dose of OVA+poly(I:C)) or 344 mm³ (4 doses of OVA+poly(I:C)). Mice in the last cohort were intentionally treated at a time when tumor size was larger in order to exemplify the beneficial effects of sequential daily immunization.

FIGS. 5A and 5B are a graphs showing HPV16 E7₄₉₋₅₇-specific CD8+ T cell responses elicited in response to a single immunization with whole Pentarix protein plus the TLR3 agonist poly(I:C). Naïve C57B1/6 mice were left untreated or were immunized with 100 μg of whole, soluble Pentarix protein admixed with 10 μg of poly(I:C), or were treated with 10 μg of poly(I:C) only (5B). Seven days post-immunization mice were euthanized and bulk splenocyte preparations were assessed by IFN-γ ELISPOT to quantitate the number of HPV 16 E7₄₉₋₅₇-specific CD8+ T cells. Briefly, splenocytes (3×10⁵ per well, triplicate wells per condition) from individual animals were stimulated overnight with either media alone or with HPV16 E7₄₉₋₅₇ peptide (10 ug/ml) or irrelevant control peptide (KAVYNFATM; SEQ ID NO: 40). Results from naïve (unimmunized) mice are included for comparison. Results are reported as the number of IFN-γ spot-forming cells per 1×10⁶ splenocytes after stimulation with media only or HPV 16 E7₄₉₋₅₇, or irrelevant peptide (10 μg/ml) (5B).

FIGS. 6A and 6B are graphs showing HPV16 E7₄₉₋₅₇-specific CD8+₊ T cell responses elicited in response to a single immunization with whole Pentarix protein plus the TLR9 agonist CpG oligonucleotide. Naïve C57B1/6 mice were left untreated or were immunized with 100 μg of whole, soluble Pentarix protein admixed with 10 μg of CpG oligo #2395 (Invivogen) or with CpG oligo only. Seven days post-immunization mice were euthanized and bulk splenocyte preparations were assessed by IFN-γ ELISPOT to quantitate the number of HPV16 E7₄₉₋₅₇-specific CD8+ T cells. Briefly, splenocytes (3×10⁵ per well, triplicate wells per condition) from individual animals were stimulated overnight with either media alone or with HPV16 E7₄₉₋₅₇ peptide (10 μg/ml) or irrelevant control peptide (KAVYNFATM; SEQ ID NO: 40). Results from naïve (unimmunized) mice are included for comparison. Results are reported as the number of IFN-γ spot-forming cells per 1×10⁶ splenocytes after stimulation with media only, HPV16 E7₄₉₋₅₇ or irrelevant peptide (10 μg/ml). The data presented in 6B are representative of three experiments; results are reported as the number of IFN-γ spot-forming cells per 1×10⁶ splenocytes+/−SD for each triplicate.

FIGS. 7A-B are graphs showing HPV16 E7₄₉₋₅₇-specific CD8+₊ T cell responses elicited in response to a single immunization with whole Pentarix protein plus the TLR3 agonist poly(I:C) or with 4 successive daily doses of Pentarix protein plus poly(I:C). Naïve C57B1/6 mice (3 per cohort for FIG. 7B) were left untreated (naïve) or were immunized one time or 4 times (daily on days 1-4) with 100 μg of whole, soluble Pentarix protein admixed with 10 μg of poly(I:C). Seven days (7A) or eight days (7B) post-immunization mice were euthanized and bulk splenocyte preparations were assessed by IFN-γ ELISPOT to quantitate the number of HPV16 E7₄₉₋₅₇-specific CD8+ T cells. Results are reported as the number of IFN-γ spot-forming cells per 1×10⁶ splenocytes after stimulation with media only or HPV16 E7₄₉₋₅₇, or irrelevant peptide (10 μg/ml) (7B). Splenocytes (3×10⁵ per well) from individual animals were stimulated overnight with either media alone or with HPV 16 E7₄₉₋₅₇ peptide (10 ug/ml) or irrelevant control peptide (KAVYNFATM; SEQ ID NO: 40). The data presented in 7B are representative of three experiments; results are reported as the number of IFN-γ spot-forming cells per 1×10⁶ splenocytes+/−SD for each triplicate.

FIG. 7C shows the results from a study where lymphocytes in spleen and peripheral blood of a mouse that was immunized for 4 successive days with 100 ug Pentarix protein plus 10 ug poly(I:C) (left two panels) or 100 ug Pentarix protein only (right panel) were stained with FITC-conjugated anti-CD8 and PE-conjugated D^{b}/16 E7₄₉₋₅₇ tetramer and analyzed by flow cytometry. Events shown are gated on CD8 lymphocytes and are representative of 4 such animals.

FIGS. 8A and 8B are a series of graphs showing immunization with whole, soluble Pentarix protein plus the TLR3 agonist poly(I:C) induces regression of large, established TC1 tumors. A, C57B1/6 mice (3 mice per cohort) were implanted with E7-expressing TC1 tumors cells (1×10⁵) on day −14 and on day 0 (when tumors reached approximately 200 mm³ in size) were left untreated (left), or were treated with 1 dose of poly(I:C) (10 μg) (middle) or 1 dose of whole, soluble Pentarix protein (100 μg) plus poly(I:C) (10 μg) (right). Tumors were measured every 2-3 days using an electronic digital caliper and size was calculated using the formula width²×length×0.5. B, Naïve C57B1/6 mice (8 per cohort) were implanted subcutaneously with 1×10⁵ E7-expressing TC-1 tumors cells. Once tumors reached an average volume of 350 mm³ mice were treated with either a single dose of Pentarix (100 ug) plus poly(I:C) (10 ug), 4 successive daily doses of Pentarix (100 ug) plus poly(I:C) (10 ug), 4 successive daily doses of poly(I:C) only (10 ug per dose) or were left untreated. Tumors were measured every 2 to 4 days with electronic calipers and tumor-bearing mice were euthanized when the tumor volume exceeded approximately 2,000 mm³ or when mice became moribund or lost >20% body weight. Data are presented as average tumor volume for all mice within a cohort (left panel) or survival (right panel).

FIG. 8C is a series of graphs showing immunization of TC 1-tumor bearing mice with Pentarix protein plus poly(I:C) elicits complete tumor regression and the establishment of E7-specific CD8+ memory cells that persist after tumor progression. Naïve C57B1/6 mice were implanted subcutaneously with 1×10⁵ E7-expressing TC-1 tumor cells. Once tumors reached an average volume of 200 mm³ mice were treated with a single dose of Pentarix (100 μg) plus poly(I:C) (10 μg) and tumors fully regressed within 15 days of immunization. A sample of peripheral blood was taken from the saphenous vein 21 days post-immunization and was stained with FITC-conjugated anti-CD8 and PE-conjugated D^{b}/16 E7₄₉₋₅₇ tetramer as well as the memory phenotype markers CD62L, CD127 and KLRG1 and analyzed by flow cytometry. Events shown are gated on CD8+ lymphocytes.

FIGS. 9A-B are a series of graphs showing immunization with whole, soluble Pentarix protein combined with poly(I:C) or CpG oligonucleotide induces regression of established TC1 tumors. A, C57B1/6 mice (4 mice per cohort) were implanted with E7-expressing TC1 tumors cells (1×10⁵) on day −21 and on day 0 were treated with 1 dose of whole, soluble Pentarix protein (100 μg) plus CpG oligo #2395 (10 μg) (upper left) or 1 dose of CpG oligo #2395 only (10 μg) (upper right) or were left untreated (lower left). Tumors were measured every 2-3 days using an electronic digital caliper and size was calculated using the formula width²×length×0.5. The upper left, upper right and lower left 3 plots show regression of tumors in individual mice, whereas the lower right plot shows combined average tumor volume measurement for each cohort. B, Naïve C57B1/6 mice (indicated number of mice per cohort) were implanted subcutaneously with 1×10⁵ E7-expressing TC-1 tumor cells. Once tumors reached an average volume of 200 mm³ mice were treated (as indicated) with either a single dose of Pentarix (100 ug) plus poly(I:C) (10 ug), a single dose of Pentarix (100 ug) plus CpG oligonucleotide (10 ug) or poly(I:C) (10 ug), CpG oligonucleotide (10 ug) or Pentarix protein (100 ug) alone or were left untreated. Tumors were measured every 2 to 4 days with electronic calipers, and data are presented as tumor volume over time for individual animals within each cohort (upper 6 panels) or as survival for all mice within a cohort (lower 2 panels). Tumor-bearing mice were euthanized when the tumor volume exceeded approximately 2,000 mm³ or when mice became moribund or lost >20% body weight). p values were calculated using the log rank (Mantel-Cox) test.

FIG. 10A is a graph showing epitope-specific CD8+ T cell responses elicited in response to a single immunization with whole Pentarix protein plus the TLR3 agonist poly(I:C). Naïve C57B1/6 mice were immunized with 100 μg of whole, soluble Pentarix protein admixed with 10 μg of poly(I:C) (Amersham). Seven days post-iimmunization mice were euthanized and bulk splenocyte preparations were assessed by IFN-γ ELISPOT to quantitate the number of CD8+ T cells specific for each of the peptides indicated. Results are reported as the number of IFN-γ spot-forming cells per 1×10⁶ splenocytes after stimulation with media only or the indicated peptide (10 μg/ml).

FIGS. 10B-D are graphs showing immunization with whole, soluble Pentarix protein plus poly(I:C) elicits immune responses against multiple genotypes of HPV. C57B1/6 (B), or HLA-A2/D^{b} transgenic mice (C) were immunized (s.c) daily for 4 successive days with 100 ug Pentarix protein combined with 10 ug poly(I:C). Eight days post-immunization mice were euthanized and bulk (B and C) or CD4-depleted splenocyte preparations (B only) were analyzed by IFN-γ ELISPOT (CD4 depletion was >99% as measured by FACS analysis post-depletion). Bulk and CD4-depleted splenocyte preparations were stimulated overnight with a panel of overlapping 15mer peptides (overlapping by 11 amino acids) that spanned the entire Pentarix protein. D, Splenocytes from C57B1/6 mice immunized (s.c) with Pentarix protein combined with 10 ug poly(I:C) were stimulated overnight with either media alone or with the minimal peptide epitopes HPV 16 E7₄₉₋₅₇, HPV31 E7₄₉₋₅₇ or irrelevant control peptide (KAVYNFATM) and analyzed by IFN-γ ELISPOT. Results are reported as the number of IFN-γ spot-forming cells per 1×10⁶ splenocytes and are representative of 3 such experiments.

FIG. 11 is a graph showing HPV16 E7₄₉₋₅₇-specific CD8+⁺ T cell responses elicited in response to a single immunization with whole Pentarix protein only (no adjuvant) or with 4 successive daily doses of Pentarix protein only (no adjuvant). Naïve C57B1/6 mice were immunized one time or 4 times (daily on days 1-4) with 100 μg of whole, soluble Pentarix protein in PBS. Seven days post-immunization mice were euthanized and bulk splenocyte preparations were assessed by IFN-γ ELISPOT to quantitate the number of HPV16 E⁷₄₉₋₅₇-specific CD8+ T cells. Results are reported as the number of IFN-γ spot-forming cells per 1×10⁶ splenocytes after stimulation with media only, HPV16 E7₄₉₋₅₇ or irrelevant negative control peptide (each at 10 μl/ml).

FIGS. 12 A-O show the amino acid sequences (SEQ ID NOs: 1-15) and the nucleotide sequences (SEQ ID NOs: 18-32) of E7 proteins from HPV16, HPV18, HPV31, HPV45, HPV52, HPV33, HPV35, HPV39, HPV51, HPV56, HPV58, HPV59, HPV68, HPV73, and HPV82, respectively.

FIGS. 12P-Q show the amino acid sequences of the Pentarix protein with (SEQ ID NO: 16) and without (SEQ ID NO: 17) an amino-terminal 6×His affinity tag.

FIGS. 12R-S show the nucleotide sequences of the Pentarix protein with (SEQ ID NO: 34) and without (SEQ ID NO: 33) an amino-terminal 6×His affinity tag.

### DETAILED DESCRIPTION

The invention provides, in part, human papillomavirus E7 antigen compounds and compositions. The compounds and compositions may be useful for treating or diagnosing human papillomavirus infection and associated conditions.

In some embodiments, compounds and compositions according to the invention are useful for targeting multiple HPV types, for example, at least two or more HPV genotypes, such as high risk HPV types. Accordingly, compounds and compositions according to the invention may be useful in inducing an immune response to one or more of the HPV types from which the HPV E7 antigens, or sequences substantially identical to the HPV E7 antigens that comprise the compounds or compositions, are derived. Such compounds and compositions with broad population coverage may be commercially useful as they are applicable to a larger group of people.

Human Papillomavirus (HPV)

By “human papillomavirus” or “HPV” is meant a virus belonging to a group of more than 100 related but genetically distinct “types” which can be broadly classified as “low risk” and “high risk.”

“Low risk” HPV types include, without limitation, HPV types HPV11, HPV40, HPV42, HPV43, HPV44, HPV54, HPV61, HPV70, HPV72, and HPV81.

“High risk” HPV types include, without limitation, HPV16, HPV18, HPV31, HPV33, HPV35, HPV39, HPV45, HPV51, HPV52, HPV56, HPV58, HPV59, HPV68, HPV73, and HPV82.

The HPV genome is generally a double-stranded circular DNA of about 7000-8000 base pairs surrounded by a protein capsid. The genome has an early (E) region encoding the early antigens E1-E7 and a late (L) region encoding the structural L1 and L2 capsid proteins. The E6 and E7 proteins are required for transformation and immortalization of infected cells and continuous expression of these proteins is required to maintain cells in a transformed state. On occasion, HPV DNA becomes integrated into the DNA of the host cell and this process is associated with the loss of several viral genes. For example, integration commonly leads to deletion of several early (E2, E4 and E5) and late (L1 and L2) genes, leaving E6 and E7 as the only viral proteins that continue to be expressed in the infected cell. HPV genome sequences have been described and are available at various public databases. Such sequences may be found, for example, at GenBank Accession numbers K02718 (HPV16), X05015 (HPV18), J04353 (HPV31), M12732 (HPV33), M62849 (HPV39), X74479 (HPV45), NC_{—}001533 (HPV51), X74481 (HPV52), X74483 (HPV56), D90400 (HPV58), NC_{—}001635/X77858 (HPV59), X67161 (HPV68), etc.

The sequences of E7 antigens from various HPV types have been described and are available at various public databases. Such sequences may be found, for example, at GenBank Accession numbers NP_{—}041326 (HPV16 E7), NP_{—}040311 (HPV18 E7), AAA46951 (HPV31 E7), AAA46959 (HPV33 E7), AAA46967 (HPV35 E7), AAA47051 (HPV39 E7), P21736 (HPV45 E7), P26558 (HPV51 E7), P36831 (HPV52 E7), P36833 (HPV56 E7), P26557 (HPV58 E7), CAA54850 (HPV59 E7), P54668 (HPV68 E7), CAA63883 (HPV73 E7), and AAK28450 (HPV82 E7), etc.

Therapeutic Indications

The compounds and compositions according to the invention may be used to treat HPV infection or a condition associated with such infection. HPV infection has been associated with a variety of conditions including, without limitation, common warts (or papillomas), cancer, etc. In general, low risk HPV types are associated with common warts (or papillomas) while high risk HPV types are associated with cancer.

By a “cancer” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumours, and carcinomas such as breast cell carcinomas and cervical carcinomas. The term cancer includes cell growths that are technically benign but which carry the risk of becoming malignant. By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor.

Accordingly, by “condition associated with HPV infection” is meant any condition, disease or disorder that has been correlated with the presence of an existing HPV infection, for example, any condition, disease or disorder that has been correlated with the presence of an existing high risk HPV infection. In some embodiments, a condition associated with HPV infection includes a condition, disease or disorder of the cervix, lower genital or anogenital tract, skin or oral cavity.

In some embodiments, a condition associated with HPV infection includes malignant and/or pre-malignant lesions of the cervix, lower genital or anogenital tract, for example, cancer of the cervix, anus, vulva, vagina, perineum, penis, etc. or pre-malignant lesions thereof. In alternative embodiments, a condition associated with HPV infection includes cancer of the lung, respiratory tract, epithelium, head and neck, breast cancer, oral cancer, etc. or pre-malignant lesions thereof.

In alternative embodiments, a condition associated with HPV infection includes a pre-malignant dysplastic condition, such as pre-cancerous cervical dysplasia, cervical intra-epithelial neoplasia (CIN) grade 1, 2, or 3, vulval intraepithelial neoplasia (VIN), vaginal intraepithelial neoplasia (VAIN), anal intraepithelial neoplasia (AlN), etc.

In some embodiments, the compounds and compositions according to the invention may be used to diagnose HPV infection. In alternative embodiments, a peptide including one or more of the sequences TSNYNIVTF (SEQ ID NO: 35), AEPDTSNYNIVTFCC (SEQ ID NO: 36) or TSNYNIVTFCCQCKS (SEQ ID NO: 37) may be used to diagnose HPV31 infection, or to determine the immune response of a compound that includes a HPV31 E7 sequence to HPV31. Alternatively, one or more of the TSNYNIVTF, AEPDTSNYNIVTFCC or TSNYNIVTFCCQCKS sequences may be used to rule out a HPV31 infection.

HPV E7 Compounds, Test Compounds, and Methods of Making Same

A compound according to the invention includes, without limitation, a polypeptide including the amino acid sequence of two or more HPV E7 antigens from different HPV genotypes, and analogues, variants, homologues and fragments thereof, as well as nucleic acid molecules encoding such polypeptides. In some embodiments, the two or more HPV E7 antigens will be capable of eliciting an immune response, such as a T cell CD8+ response, against the different HPV genotypes from which they are derived.

A “protein,” “peptide” or “polypeptide” is any chain of two or more amino acids, including naturally occurring or non-naturally occurring amino acids or amino acid analogues, regardless of post-translational modification (e.g., glycosylation or phosphorylation). An “amino acid sequence”, “polypeptide”, “peptide” or “protein” of the invention may include peptides or proteins that have abnormal linkages, cross links and end caps, non-peptidyl bonds or alternative modifying groups. Such modifications are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain. Alternatively, the modifying group can be coupled to a side chain of at least one amino acid residue of a peptidic structure, or to a peptidic or peptidomimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

The terms “nucleic acid” or “nucleic acid molecule” encompass both RNA (plus and minus strands) and DNA, including cDNA, genomic DNA, and synthetic (e.g., chemically synthesized) DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. One example of a modified RNA included within this term is phosphorothioate RNA. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). Thus a “cDNA clone” means a duplex DNA sequence complementary to an RNA molecule of interest, carried in a cloning vector. By “complementary” is meant that two nucleic acids, e.g., DNA or RNA, contain a sufficient number of nucleotides which are capable of forming Watson-Crick base pairs to produce a region of double-strandedness between the two nucleic acids. Thus, adenine in one strand of DNA or RNA pairs with thymine in an opposing complementary DNA strand or with uracil in an opposing complementary RNA strand. It will be understood that each nucleotide in a nucleic acid molecule need not form a matched Watson-Crick base pair with a nucleotide in an opposing complementary strand to form a duplex. A nucleic acid molecule is “complementary” to another nucleic acid molecule if it hybridizes, under conditions of high stringency, with the second nucleic acid molecule. A nucleic acid molecule according to the invention includes both complementary molecules.

In some embodiments, a compound according to the invention includes, without limitation, a polypeptide including an amino acid sequence substantially identical to the amino acid sequence of two or more E7 antigens from different HPV types, such as HPV16, HPV18, HPV31, HPV33, HPV35, HPV39, HPV45, HPV51, HPV52, HPV56, HPV58, HPV59, HPV68, HPV73, or HPV82, or a nucleic acid molecule encoding such a polypeptide.

In alternative embodiments, a compound according to the invention includes, without limitation, a polypeptide including an amino acid sequence substantially identical to the amino acid sequence of three or more E7 antigens from different HPV types, such as HPV types, such as HPV16, HPV18, HPV31, HPV33, HPV35, HPV39, HPV45, HPV51, HPV52, HPV56, HPV58, HPV59, HPV68, HPV73, or HPV82, or a nucleic acid molecule encoding such a polypeptide.

In alternative embodiments, a compound according to the invention includes, without limitation, a polypeptide including an amino acid sequence substantially identical to the amino acid sequence of four or more E7 antigens from different HPV types, such as HPV types, such as HPV16, HPV18, HPV31, HPV33, HPV35, HPV39, HPV45, HPV51, HPV52, HPV56, HPV58, HPV59, HPV68, HPV73, or HPV82, or a nucleic acid molecule encoding such a polypeptide.

In alternative embodiments, a compound according to the invention includes, without limitation, a polypeptide including an amino acid sequence substantially identical to the amino acid sequence of five or more E7 antigens from different HPV types, such as HPV16, HPV18, HPV31, HPV33, HPV35, HPV39, HPV45, HPV51, HPV52, HPV56, HPV58, HPV59, HPV68, HPV73, or HPV82, or a nucleic acid molecule encoding such a polypeptide.

In alternative embodiments, a compound according to the invention includes, without limitation, a polypeptide including an amino acid sequence substantially identical to the amino acid sequence of five E7 antigens from different HPV types, such as HPV16, HPV18, HPV31, HPV45, and HPV52, or a nucleic acid molecule encoding such a polypeptide.

In some embodiments, a compound according to the invention includes, without limitation, a polypeptide including an amino acid sequence substantially identical to the amino acid sequence of two or more of SEQ ID NOs: 1-15.

In some embodiments, a compound according to the invention includes, without limitation, a polypeptide including an amino acid sequence substantially identical to the amino acid sequence of three or more of SEQ ID NOs: 1-15.

In some embodiments, a compound according to the invention includes, without limitation, a polypeptide including an amino acid sequence substantially identical to the amino acid sequence of four or more of SEQ ID NOs: 1-15.

In some embodiments, a compound according to the invention includes, without limitation, a polypeptide including an amino acid sequence substantially identical to the amino acid sequence of five or more of SEQ ID NOs: 1-15.

For example, a compound according to the invention includes, without limitation, a polypeptide including an amino acid sequence substantially identical to the amino acid sequence of SEQ ID NO: 16 or 17.

In some embodiments, a compound according to the invention includes, without limitation, a nucleic acid molecule including a nucleotide sequence substantially identical to the nucleotide sequence of two or more of SEQ ID NOs: 18-32.

In some embodiments, a compound according to the invention includes, without limitation, a nucleic acid molecule including a nucleotide sequence substantially identical to the nucleotide sequence of three or more of SEQ ID NOs: 18-32.

In some embodiments, a compound according to the invention includes, without limitation, a nucleic acid molecule including a nucleotide sequence substantially identical to the nucleotide sequence of four or more of SEQ ID NOs: 18-32.

In some embodiments, a compound according to the invention includes, without limitation, a nucleic acid molecule including a nucleotide sequence substantially identical to the nucleotide sequence of all five of SEQ ID NOs: 18-22.

For example, a compound according to the invention includes, without limitation, a nucleic acid molecule including a nucleotide sequence substantially identical to the nucleotide sequence of SEQ ID NOs: 33 or 34.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy or substantially reduce T cell recognition and/or HLA binding of the polypeptide expressed by the amino acid sequence or encoded by the nucleic acid molecule. Such a sequence can be any value from 50% to 99%, or more generally at least 50%, 55% or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

In some embodiments, a compound according to the invention includes a compound that is substantially identical to a native HPV E7 antigen sequence. Accordingly, sequences for use in the compounds according to the invention may include sequences that are substantially identical to any of SEQ ID NOs: 1-34, or to any other HPV E7 sequences.

It is to be understood that the individual E7 antigen sequences may occur in any order in the amino acid or nucleotide sequence of a compound according to the invention, as long as at least two or more E7 antigen sequences from different HPV genotypes are present in a single molecule. In some embodiments, 3, 4, 5, 6, 7, 8, 9 or 10 or more different HPV E7 antigens from different HPV types may be used in a compound according to the invention.

Exemplary HPV E7 antigen sequence ordering include those set forth in Table 1.

TABLE 1
Exemplary permutations of E7 sequences from 5 HPV types

| | | |
|---|---|---|
| 16, 18, 31, 45, 52 | 16, 18, 31, 52, 45 | 16, 18, 45, 31, 52 |
| 16, 18, 45, 52, 31 | 16, 18, 52, 31, 45 | 16, 18, 52, 45, 31 |
| 16, 31, 18, 45, 52 | 16, 31, 18, 52, 45 | 16, 31, 45, 18, 52 |
| 16, 31, 45, 52, 18 | 16, 31, 52, 18, 45 | 16, 31, 52, 45, 18 |
| 16, 45, 18, 31, 52 | 16, 45, 18, 52, 31 | 16, 45, 31, 18, 52 |
| 16, 45, 31, 52, 18 | 16, 45, 52, 18, 31 | 16, 45, 52, 31, 18 |
| 16, 52, 18, 31, 45 | 16, 52, 18, 45, 31 | 16, 52, 31, 18, 45 |
| 16, 52, 31, 45, 18 | 16, 52, 45, 18, 31 | 16, 52, 45, 31, 18 |
| 18, 16, 31, 45, 52 | 18, 16, 31, 52, 45 | 18, 16, 45, 31, 52 |
| 18, 16, 45, 52, 31 | 18, 16, 52, 31, 45 | 18, 16, 52, 45, 31 |
| 18, 31, 16, 45, 52 | 18, 31, 16, 52, 45 | 18, 31, 45, 16, 52 |
| 18, 31, 45, 52, 16 | 18, 31, 52, 16, 45 | 18, 31, 52, 45, 16 |
| 18, 45, 16, 31, 52 | 18, 45, 16, 52, 31 | 18, 45, 31, 16, 52 |
| 18, 45, 31, 52, 16 | 18, 45, 52, 16, 31 | 18, 45, 52, 31, 16 |
| 18, 52, 16, 31, 45 | 18, 52, 16, 45, 31 | 18, 52, 31, 16, 45 |
| 18, 52, 31, 45, 16 | 18, 52, 45, 16, 31 | 18, 52, 45, 31, 16 |
| 31, 16, 18, 45, 52 | 31, 16, 18, 52, 45 | 31, 16, 45, 18, 52 |
| 31, 16, 45, 52, 18 | 31, 16, 52, 18, 45 | 31, 16, 52, 45, 18 |
| 31, 18, 16, 45, 52 | 31, 18, 16, 52, 45 | 31, 18, 45, 16, 52 |
| 31, 18, 45, 52, 16 | 31, 18, 52, 16, 45 | 31, 18, 52, 45, 16 |
| 31, 45, 16, 18, 52 | 31, 45, 16, 52, 18 | 31, 45, 18, 16, 52 |
| 31, 45, 18, 52, 16 | 31, 45, 52, 16, 18 | 31, 45, 52, 18, 16 |
| 31, 52, 16, 18, 45 | 31, 52, 16, 45, 18 | 31, 52, 18, 16, 45 |
| 31, 52, 18, 45, 16 | 31, 52, 45, 16, 18 | 31, 52, 45, 18, 16 |
| 45, 16, 18, 31, 52 | 45, 16, 18, 52, 31 | 45, 16, 31, 18, 52 |
| 45, 16, 31, 52, 18 | 45, 16, 52, 18, 31 | 45, 16, 52, 31, 18 |
| 45, 18, 16, 31, 52 | 45, 18, 16, 52, 31 | 45, 18, 31, 16, 52 |
| 45, 18, 31, 52, 16 | 45, 18, 52, 16, 31 | 45, 18, 52, 31, 16 |
| 45, 31, 16, 18, 52 | 45, 31, 16, 52, 18 | 45, 31, 18, 16, 52 |
| 45, 31, 18, 52, 16 | 45, 31, 52, 16, 18 | 45, 31, 52, 18, 16 |
| 45, 52, 16, 18, 31 | 45, 52, 16, 31, 18 | 45, 52, 18, 16, 31 |
| 45, 52, 18, 31, 16 | 45, 52, 31, 16, 18 | 45, 52, 31, 18, 16 |
| 52, 16, 18, 31, 45 | 52, 16, 18, 45, 31 | 52, 16, 31, 18, 45 |
| 52, 16, 31, 45, 18 | 52, 16, 45, 18, 31 | 52, 16, 45, 31, 18 |
| 52, 18, 16, 31, 45 | 52, 18, 16, 45, 31 | 52, 18, 31, 16, 45 |
| 52, 18, 31, 45, 16 | 52, 18, 45, 16, 31 | 52, 18, 45, 31, 16 |
| 52, 31, 16, 18, 45 | 52, 31, 16, 45, 18 | 52, 31, 18, 16, 45 |
| 52, 31, 18, 45, 16 | 52, 31, 45, 16, 18 | 52, 31, 45, 18, 16 |
| 52, 45, 16, 18, 31 | 52, 45, 16, 31, 18 | 52, 45, 18, 16, 31 |
| 52, 45, 18, 31, 16 | 52, 45, 31, 16, 18 | 52, 45, 31, 18, 16 |

In some embodiments, the E7 antigen sequences are “naturally occurring” or “native” i.e., isolated from a natural source rather than artificially modified. Such sources may include, without limitation, biological samples (e.g., blood, serum, plasma, semen, mucus, urine, oral, vaginal and cervical fluids, gynecological sample, biopsies, etc.) obtained from infected subjects or from other source.

In alternative embodiments, compounds can be prepared by, for example, replacing, deleting, or inserting an amino acid residue at any position of the E7 antigen sequences from any HPV type or polypeptide as described herein, with other conservative amino acid residues, i.e., residues having similar physical, biological, or chemical properties, and for example screening for the ability of the compound to elicit a CD8+ T cell response as described herein or known in the art. In some embodiments, fragments of native E7 antigens are contemplated within the scope of the invention, as long as the fragments do not exhibit no or substantially reduced T cell recognition and/or HLA binding.

As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Ornithine.

In some embodiments, conserved amino acid substitutions may be made where an amino acid residue is substituted for another having a similar hydrophilicity value (e.g., within a value of plus or minus 2.0, or plus or minus 1.5, or plus or minus 1.0, or plus or minus 0.5), where the following may be an amino acid having a hydropathic index of about −1.6 such as Tyr (−1.3) or Pro (−1.6) assigned to the amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (O); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conservative amino acid substitutions may be made where an amino acid residue is substituted for another having a similar hydropathic index (e.g., within a value of plus or minus 2.0, or plus or minus 1.5, or plus or minus 1.0, or plus or minus 0.5). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conservative amino acid substitutions may be made using publicly available families of similarity matrices (73-79). The PAM matrix is based upon counts derived from an evolutionary model, while the Blosum matrix uses counts derived from highly conserved blocks within an alignment. A similarity score of above zero in either of the PAM or Blosum matrices may be used to make conservative amino acid substitutions.

In alternative embodiments, conservative amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (80). Genetically encoded hydrophobic amino acids include Gly, Ala, Phe, Val, Leu, Ile, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (C₁-C₆)alkyl, substituted (C₁-C₆)alkyl, (C₁-C₆)alkenyl, substituted (C₁-C₆)alkenyl, (C₁-C₆)alkynyl, substituted (C₁-C₆)alkynyl, (C₅-C₂₀) aryl, substituted (C₅-C₂₀) aryl, (C₆-C₂₆)alkaryl, substituted (C₆-C₂₆)alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Trp, while non-genetically encoded aromatic amino acids include phenylglycine, 2-napthylalanine, beta-2-thienylalanine, 1,2,3,4-tetrahydro-isoquinoline-3-carboxylic acid, 4-chlorophenylalanine, 2-fluorophenylalanine-3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids ornithine, 2,3,-diaminopropionic acid, 2,4-diaminobutyric acid, and homoarginine.

Accordingly, conservative substitutions include, without limitation, the following substitutions:

| Original Residue | Exemplary Substitutions | Preferred Substitutions |
|---|---|---|
| Ala (A) | val; leu; ile | val |
| Arg (R) | lys; gln; asn | lys |
| Asn (N) | gln; his; asp, lys; gln | arg |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-im-benzylhistidine for the imidazole nitrogen of histidine. Peptide analogues also include amino acids that have been chemically altered, for example, by methylation, by amidation of the C-terminal amino acid by an alkylamine such as ethylamine, ethanolamine, or ethylene diamine, or acylation or methylation of an amino acid side chain (such as acylation of the epsilon amino group of lysine). Peptide analogues can also include replacement of the amide linkage in the peptide with a substituted amide (for example, groups of the formula —C(O)—NR, where R is (C₁-C₆)alkyl, (C₁-C₆)alkenyl, (C₁-C₆)alkynyl, substituted (C₁-C₆)alkyl, substituted (C₁-C₆)alkenyl, or substituted (C₁-C₆)alkynyl) or isostere of an amide linkage (for example, —CH₂NH—, —CH₂S, —CH₂CH₂—, —CH═CH— (cis and trans), —C(O)CH₂—, —CH(OH)CH₂—, or —CH₂SO—).

The compound can be covalently linked, for example, by polymerisation or conjugation, to form homopolymers or heteropolymers. Spacers and linkers, typically composed of small neutral molecules, such as amino acids that are uncharged under physiological conditions, can be used. Linkages can be achieved in a number of ways. For example, cysteine residues can be added at the peptide termini, and multiple peptides can be covalently bonded by controlled oxidation. Alternatively, heterobifunctional agents, such as disulfide/amide forming agents or thioether/amide forming agents can be used. The compound can also be linked to another compound that can modulate an immune response. The compound can also be constrained, for example, by having cyclic portions.

Polypeptides, peptides or peptide analogues can be synthesised by standard chemical techniques, for example, by automated synthesis using solution or solid phase synthesis methodology. Automated peptide synthesisers are commercially available and use techniques well known in the art. Polypeptides, peptides and peptide analogues can also be prepared from their corresponding nucleic acid molecules using recombinant DNA technology using standard methods such as those described in, for example, Sambrook, et al. (81) or Ausubel et al. (82).

In some embodiments, a nucleic acid molecule may be operably linked. By “operably linked” is meant that a gene and a regulatory sequence(s) are connected in such a way as to permit gene expression when the appropriate molecules (e.g., transcriptional activator proteins) are bound to the regulatory sequence(s). Such operably linked sequences may be in the form of vectors or expression constructs that can be transformed or transfected into host cells for expression. Any suitable vector can be used such as for example pET15b (ampicillin resistant) or pET24a (kanamycin resistant).

The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

Compounds identified as being useful may be subsequently analyzed using a TC1 model, or any other animal model for HPV infection.

Pharmaceutical & Veterinary Compositions, Dosages, and Administration

Compounds of the invention can be provided alone or in combination with other compounds (for example, nucleic acid molecules, small molecules, peptides, or peptide analogues), in the presence of a liposome, an adjuvant, or any carrier, such as a pharmaceutically acceptable carrier, in a form suitable for administration to mammals, for example, humans, cattle, sheep, etc.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Conventional pharmaceutical practice may be employed to provide suitable formulations or compositions to administer the compounds to subjects suffering from HPV infection or presymptomatic for a condition associated with HPV infection. Any appropriate route of administration may be employed, for example, parenteral, intravenous, subcutaneous, intramuscular, intracranial, intraorbital, ophthalmic, intraventricular, intracapsular, intraspinal, intrathecal, intracisternal, intraperitoneal, intranasal, aerosol, topical, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences” (83). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. In general, the compounds are administered to an individual in an amount sufficient to stop or slow a condition associated with HPV infection, or to treat HPV infection, depending on the disorder.

In the case of vaccine formulations, an effective amount of a compound of the invention can be provided, alone or in combination with other compounds, with an immunological adjuvant, for example, Freund's incomplete adjuvant, dimethyldioctadecylammonium hydroxide, or aluminum hydroxide.

In alternative embodiments, a compound according to the invention may be provided in combination with an adjuvant selected from a Toll-like receptor (TLR) agonist, such as a TLR3 agonist (e.g., poly(I:C) and derivatives thereof, polyA:U and derivatives thereof, synthetic RNA molecules, naturally occurring RNA molecules, double-stranded RNAs, microbial nucleic acids etc.) or a TLR9 agonist (e.g., a CpG containing oligonucleotide, microbial nucleic acids, etc.), an interferon-alpha, an agonist of the 4-1BB receptor, an agonist of the CD40 receptor, or an anti-CD40 antibody.

The compound may also be linked with a carrier or other molecule, such as bovine serum albumin or keyhole limpet hemocyanin to enhance immunogenicity. In some embodiments, the compound may be provided with calreticulin, Mycobacterium tuberculosis heat shock protein (HSP70), ubiquitin, bacterial toxin, cytokine (such as an interleukin), imidazoquimolines, etc.

In some embodiments, compounds or compositions according to the invention may be provided in a kit, optionally with a carrier and/or an adjuvant, together with instructions for use.

An “effective amount” of a compound according to the invention includes a therapeutically effective amount, immunologically effective amount, or a prophylactically effective amount. A “therapeutically effective amount” refers to an amount effective, at dosages and for periods of time necessary, to achieve the desired therapeutic result, such as treatment of HPV infection or a condition associated with such infection. The outcome of the treatment may for example be measured by a decrease in HPV viremia, inhibition of viral gene expression, delay in development of a pathology associated with HPV infection, stimulation of the immune system, or any other method of determining a therapeutic benefit. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. By “immunogenically effective amount” is meant an amount effective, at dosages and for periods of time necessary, to achieve the desired immune result, such as stimulation or elicitation of an immune response, such as a T cell CD8+ response. In some embodiments, by “stimulation of an immune response” or “stimulating an immune response” is meant an increase in the measured immune response, such as a T cell CD8+ response, of any value between about 5% and about 95%, or between about 10% and about 90%, or between about 30% and about 60%, or over 100% increase when compared with a control or reference sample or compound. In alternative embodiments, by “stimulation of an immune response” or “stimulating an immune response” is meant an increase in the measured immune response, such as a T cell CD8+ response, of any value between about a 2-fold and about a 1000-fold, or about a 10-fold to about a 500-fold, or about a 30-fold to about a 100-fold, or more than a 1000-fold increase when compared with a control or reference sample or compound. A “prophylactically effective amount” refers to an amount effective, at dosages and for periods of time necessary, to achieve the desired prophylactic result, such as prevention of onset of a condition associated with HPV infection. Typically, a prophylactic dose is used in subjects prior to or at an earlier stage of disease, so that a prophylactically effective amount may be less than a therapeutically effective amount. A suitable range for effective amounts of a compound may for example be any integer from 0.1 nM-0.1M, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In alternative embodiments, a composition including a compound according to the invention, in combination with a TLR agonist or other adjuvant, may be provided in a “cluster” dosing regimen to elicit an enhanced CD8+ T cell response. By a “cluster” dosing regimen is meant administration of the composition over a short period of time i.e., less than about 14 days, for example, about 1 day to about 4, 5, 6, 7 or 8 days. In some embodiments, a cluster dosing regimen includes administration of multiple daily doses of the composition over a short period of time i.e., less than about 14 days, for example, about 1 day to about 4, 5, 6, 7 or 8 days.

If desired, treatment with a compound according to the invention may be combined with more traditional and existing therapies for HPV infection or a condition associated with such infection. For example, a compound according to the invention may be provided in combination with radiation therapy, chemotherapy or surgery (e.g., LEEP) as appropriate.

Compounds according to the invention may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

The present invention will be further illustrated in the following examples.

### Example 1

### Preparation and Purification of Pentarix Protein

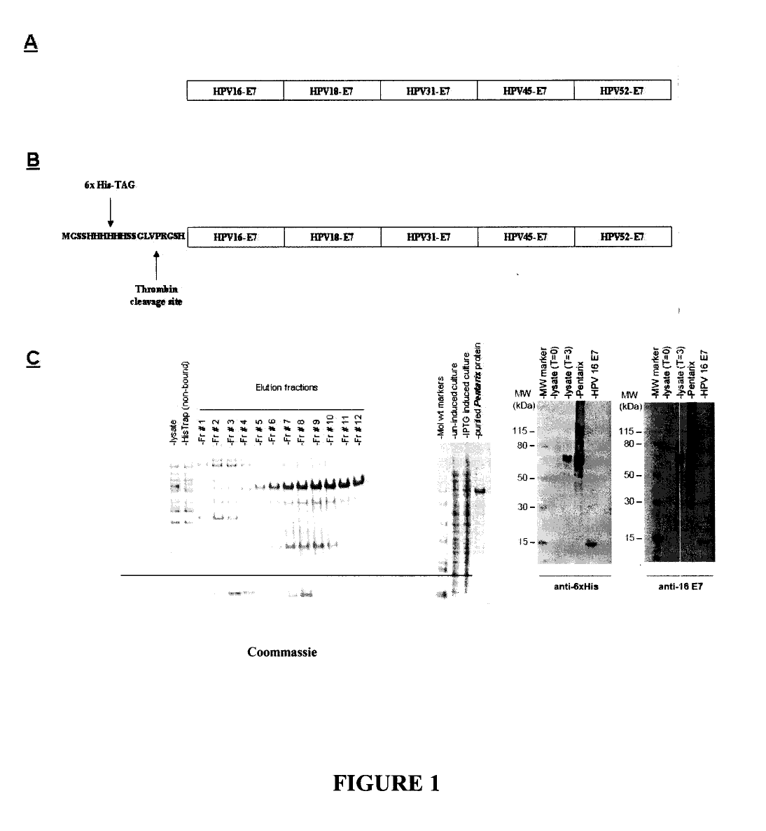

The E7 oncoproteins of HPV strains 16, 18, 31, 45 and 52 were produced as a full length recombinant protein (termed “Pentarix”) in E. coli (FIG. 1A). In one study, a single contiguous DNA comprising the complete E7 protein from each of HPV 16, 18, 31, 45 and 52 plus an amino-terminal 6×HIS affinity TAG and thrombin cleavage site was produced as a synthetic DNA construct (FIG. 1B). The multi-gene sequence was subsequently cloned into the expression vectors pET17 and pET24a (Invitrogen) and full length protein (Pentarix) with a cleavable 6×HIS affinity TAG was expressed in E. coli genotype BL21 (DE3) pLysS.

Briefly, 5 ml of LB media containing ampicillin (100 μg/ml) plus chloramphenicol (34 μg/ml) in the case of pET17 constructs, or containing kanamycin (34 ug/ml) plus chloramphenicol (34 ug/ml) in the case of pET24a constructs, was inoculated with a single colony of recombinant E. coli and allowed to grow to saturation overnight. The next morning the 5 ml culture was used to inoculate 1000 ml of LB media containing ampicillin (100 μg/ml) plus chloramphenicol (34 μg/ml in the case of pET17 constructs, or containing kanamycin (34 ug/ml) plus chloramphenicol (34 ug/ml) in the case of pET24a constructs. When the growth in the 1000 ml culture reached OD₆₀₀ of between 0.2 and 0.4, IPTG was added to the culture to a final concentration of 2 mM and growth was continued for another 2 to 3 hours to allow for expression of recombinant protein. Bacteria were subsequently pelleted by centrifugation and resuspended in 30 ml lysis buffer (20 mM sodium phosphate, pH 7.4, 500 mM sodium chloride, 10 mM imidazole, 6 M urea, 1 mM DTT). Bacteria were lysed by two successive cycles of freezing and thawing followed by four successive cycles of sonication (30 seconds per cycle). Debris and insoluble protein was removed by centrifugation for 15 minutes at 15,000 rpm to render a soluble lysate solution. Pentarix protein with a cleavable 6× affinity tag was purified from soluble lysate by passage over an affinity column (HisTrap HP, GE Healthcare) attached to an AKTA column chromatography system. After extensive washing with lysis buffer, Pentarix protein with a cleavable 6× affinity tag was eluted from the column using lysis buffer containing 500 mM imidazole. Elution fractions containing Pentarix protein with a cleavable 6× affinity tag were then pooled and dialyzed against 4 changes of tissue culture grade phosphate buffered saline to render the final solution of Pentarix protein. Protein expression and purification was monitored by running various in process and final fractions on SDS-PAGE and visualizing proteins via Coomassie Blue staining or Western blot using anti-6×HIS Tag antibody (ABM) or anti-HPV 16E7 antibody (Invitrogen) (FIG. 1C). The purified protein was fully soluble in PBS and migrated on SDS-PAGE gel in accordance with its predicted molecular weight of 59,037 Da (FIG. 1C).

### Example 2

### CD8 Responses to Exogenous Protein Antigen

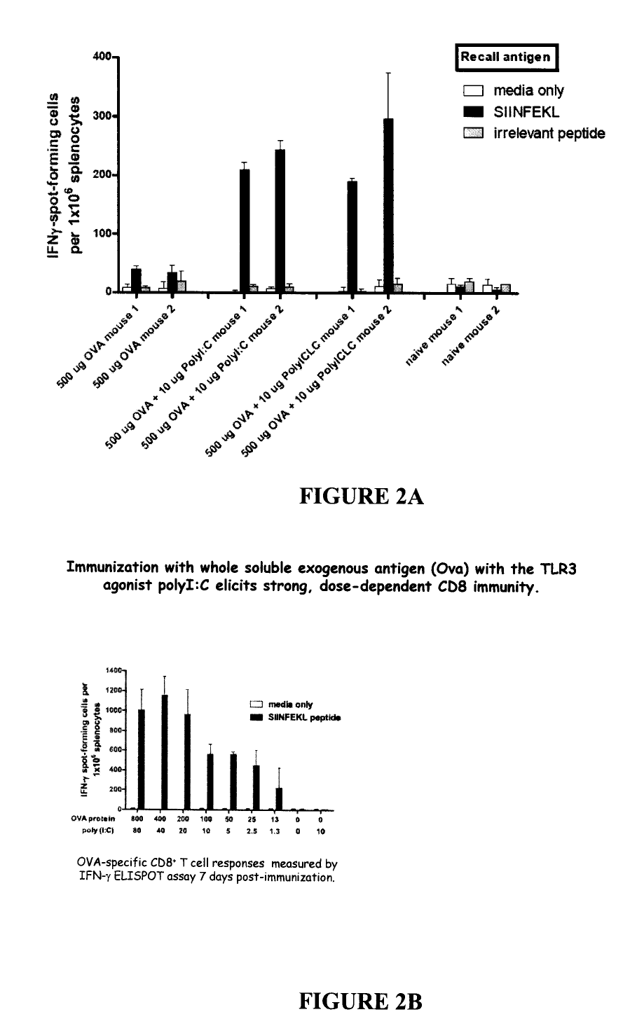

Studies to determine OVA₂₅₇₋₂₆₄-specific CD8+₊ T cell responses elicited in response to immunization with whole exogenous OVA protein plus the TLR3 agonists poly(I:C) or polyIC/LC were performed. Naïve C57B1/6 mice (2 mice per condition) were immunized with whole OVA protein (500 μg) plus or minus poly(I:C) (10 μg) or polyIC/LC (10 μg/ml). Seven days post-immunization mice were euthanized and the number of OVA₂₅₇₋₂₆₄ (SIINFEKL)-specific CD8+ T cells in bulk splenocytes of immunized mice were quantitated by IFN-γ ELISPOT. Results are reported as the number of IFN-γ spot-forming cells per 1×10⁶ splenocytes after stimulation with media only, SIINFEKL peptide (10 μg/ml) or irrelevant H2 Db-binding peptide (10 μg/ml). The results suggest that poly(I:C) and polyIC/LC have comparable adjuvant activity in vivo (FIG. 2A).

In another study, naïve C57B1/6 mice (2 mice per dose, 18 mice in total) were immunized with the indicated amounts of whole, soluble OVA protein plus poly(I:C). Seven days post-immunization mice were euthanized and bulk splenocyte preparations were assessed by IFN-γ ELISPOT. Splenocytes (3×10⁵ per well, triplicate wells per condition) from individual animals were stimulated overnight with either media alone or with SIINFEKL peptide (10 ug/ml), and results are reported as the number of IFN-γ spot-forming cells per 1×10⁶ splenocytes+/−SD for each cohort (FIG. 2B).

### Example 3

### Dosing Regimens

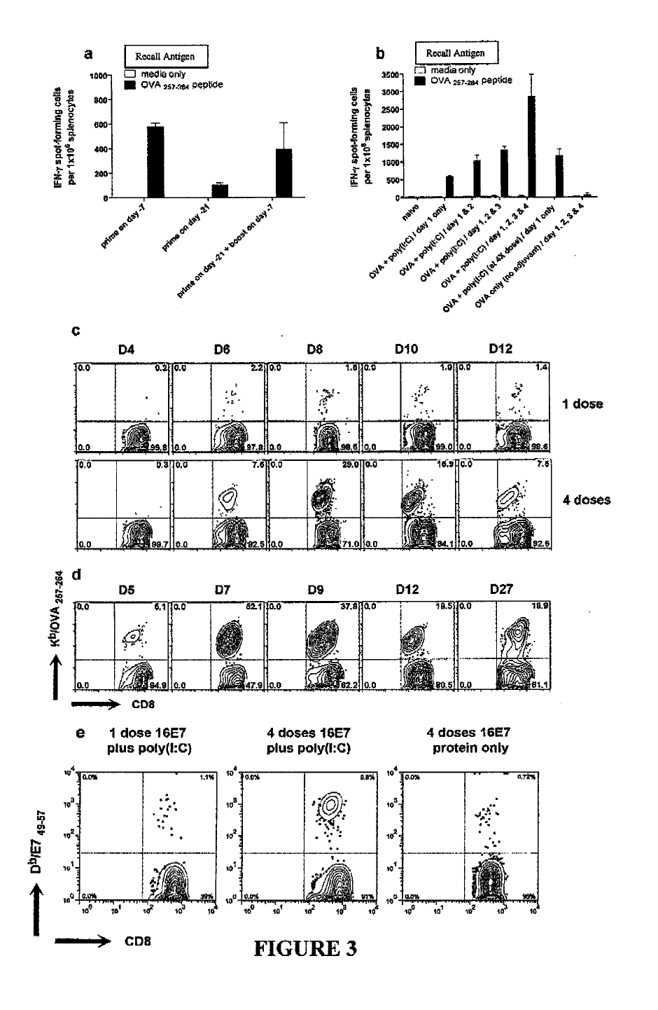

OVA₂₅₇₋₂₆₄-specific CD8+ T cell responses elicited in response to long or short interval (cluster) homologous prime-boost immunization were determined as follows. Naïve C57B1/6 mice (2 mice per condition) were immunized with whole OVA protein (100 μg) plus poly(I:C) (10 μg) at day −7, day −21 or day −7 and day −21 and were euthanized at day 0. The number of OVA₂₅₇₋₂₆₄-specific CD8+ T cells in bulk splenocytes of immunized mice were quantitated by IFN-γ ELISPOT. Naïve C57B1/6 mice (3 mice per condition) were also immunized with the indicated number of sequential daily doses of whole, soluble OVA protein (100 μg) admixed with poly(I:C) (10 μg). One additional group of mice received a single immunization that was equivalent to four times the normal daily dose (i.e. 400 μg of OVA protein plus 40 μg of poly(I:C)). Seven days after the first immunization mice were euthanized and bulk splenocyte preparations were assessed by IFN-γ ELISPOT to quantitate the number of OVA₂₅₇₋₂₆₄-specific CD8+ T cells. The results in FIGS. 3a and b are reported as the number of IFN-γ spot-forming cells per 1×10⁶ splenocytes after stimulation with media only or SIINFEKL peptide (10 μg/ml). Naïve C57B1/6 mice were also immunized with one dose or four consecutive daily doses of whole, soluble OVA protein (100 μg) plus poly(I:C) (10 mg) as indicated in FIG. 3c. Mice that received 4 four consecutive daily doses of OVA protein plus poly(I:C) were reimmunized with another four consecutive daily doses of the same, starting at day 47 after imitation of the first round of immunization. Peripheral blood was obtained from the saphenous vein of individual mice that were serially bled on the indicated days post-immunization. RBC in peripheral blood were lysed and lymphocytes were stained with FITC-conjugated anti-CD8 and PE-conjugated H-2 Kb/OVA₂₅₇₋₂₆₄ tetramer and analyzed by flow cytometry. Events shown in FIGS. 3c and d were gated on CD8+ lymphocytes and were from a representative single animal to allow precise monitoring of the evolution of the antigen-specific T cell responses within a given animal over time. After this second round of vaccination, OVA-specific CD8+ T cells expanded to levels that were even higher than what was achieved after primary immunization, reaching 52% of peripheral CD8+ T cells within 7 days of secondary immunization (FIG. 3d). Accordingly, the secondary response elicited at a later time point was considerably stronger than the primary response elicited by cluster vaccination.

In another study, as observed with the model antigen OVA, immunization with four doses of HPV16 E7 protein plus poly(I:C) elicited a robust CD8 immune response compared to immunization with HPV16 E7 protein alone (FIG. 3e).

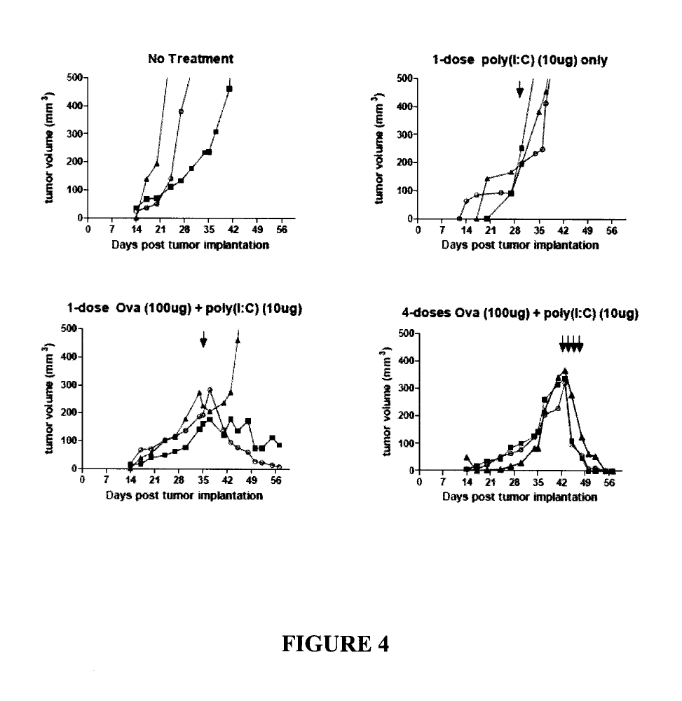

In another study, C57B1/6 mice (3 mice per cohort) were implanted with OVA-expressing EG7 tumors cells (1×10⁵) on day 0 and were left untreated, or were treated with 1 dose of poly(I:C) (10 μg), 1 dose of whole, soluble OVA protein (100 μg) plus poly(I:C) (10 μg) or four sequential daily doses of whole, soluble OVA protein (100 μg) plus poly(I:C) (10 μg) for specified periods of time, as shown in FIG. 4. Average tumor volume at time of treatment for each group was 224 mm³ (poly(I:C) only), 194 mm³ (1 dose of OVA+poly(I:C)) or 344 mm³ (4 doses of OVA+poly(I:C)). Mice in the last cohort were intentionally treated at a time when tumor size was larger in order to exemplify the beneficial effects of sequential daily immunization.

### Example 4

### Immunization with Single or Multiple Doses of Pentarix Protein

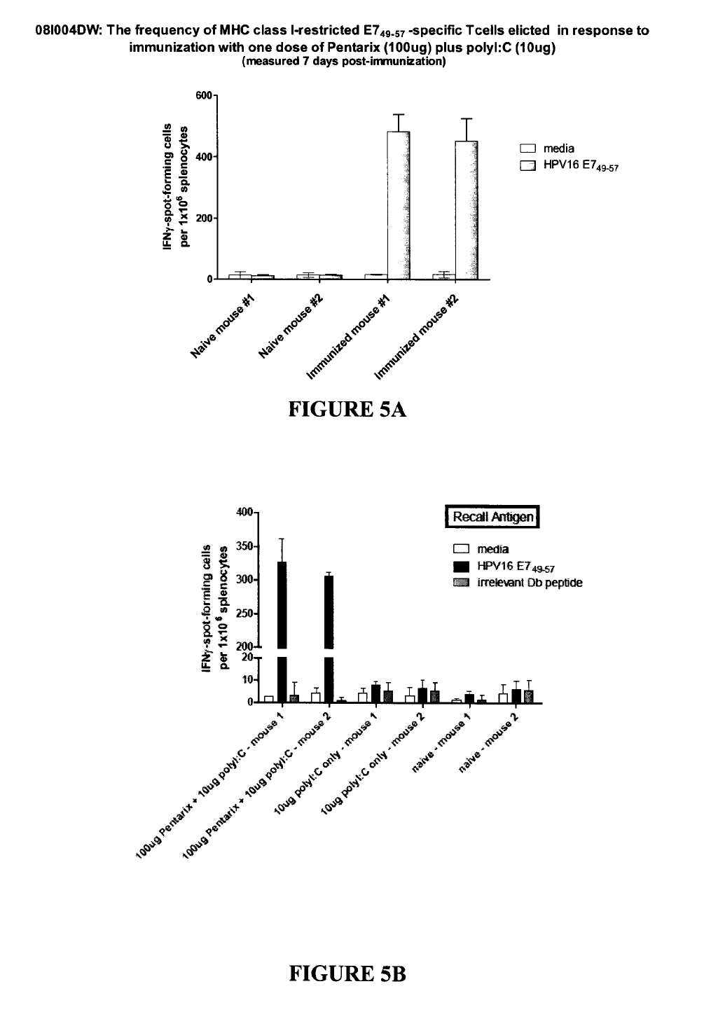

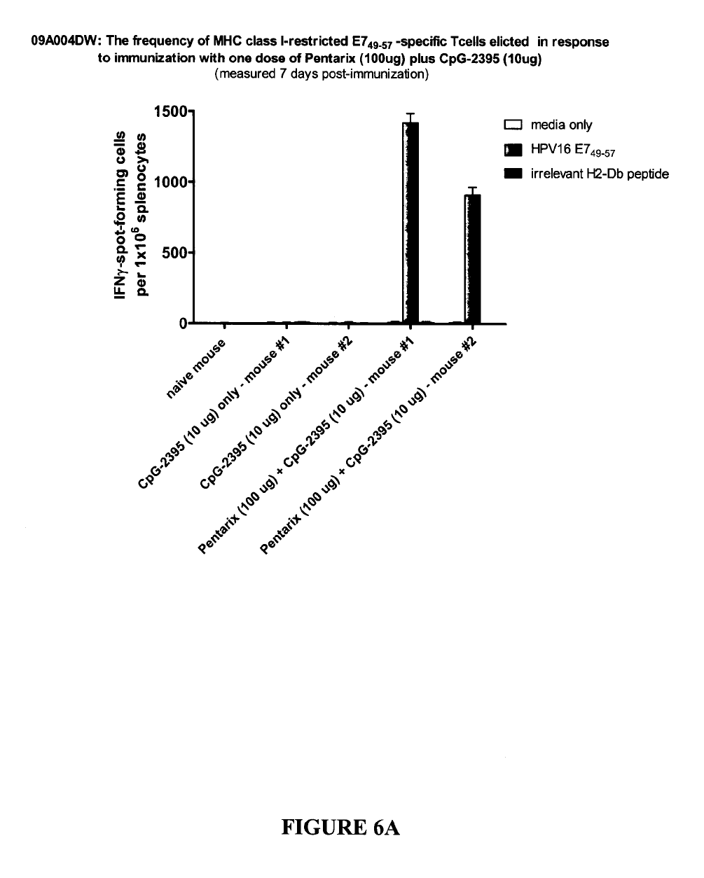

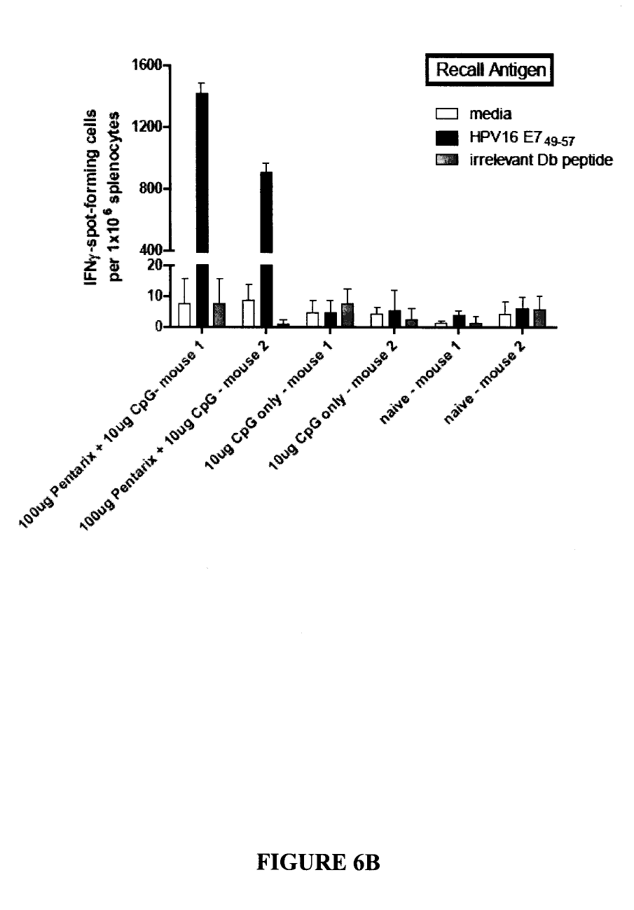

Mice (naïve C57B1/6) were left untreated or were immunized subcutaneously with 100 μg of recombinant Pentarix protein admixed with 10 μg of the TLR3 agonist polyI-C (Amersham or Sigma) (FIG. 5A) or the TLR9 agonist CpG-2395 (oligo #2395, Invivogen), or with CpG oligo only (FIG. 6A). Seven days post-immunization mice were euthanized and spleens were excised. Single cell suspensions of splenocytes were prepared in 10 ml of cRPMI (RPMI 1640, 10% FCS, 2 mM L-glutamine, 50 uM 2-mercaptoethanol, 10 mM HEPES and 10 mM sodium pyruvate) by mashing spleens through a 70 uM filter using the plunger from a 5 ml syringe. ELISPOT plates (MSIP, Millipore) were pre-coated overnight with 10 μg/ml anti-IFN-γ capture antibody (AN18-Mabtech) and then blocked for 2 hours at 37° C. with cRPMI. Splenocytes (3×10⁵ cells per well) were plated in triplicate in the absence of any stimulus (media only), in the presence of 10 μg/ml of the H-2D^{b} restricted E7₄₉₋₅₇ peptide from HPV16, or an irrelevant H2-D^{b}-binding control peptide (KAVYNFATC). After overnight incubation at 37° C., to ELISPOT plates were washed and incubated for 2 hours at 37° C. with 1 μg/mlbiotinylated anti-mouse IFN-γ (mAb R4-6A2, Mabtech) followed by development with Vectastain ABC Elite kit and Vectastain AEC substrate reagent according to manufacturers' instructions (Vector Labs). Spots were quantitated using a commercial ELISPOT counting service (Zellnet). Results are presented as the number of IFN-γ spot forming cells per 1×10⁶ splenocytes when cultured in the presence of media only, media plus HPV16 E7₄₉₋₅₇ peptide or irrelevant peptide (10 μg/ml).

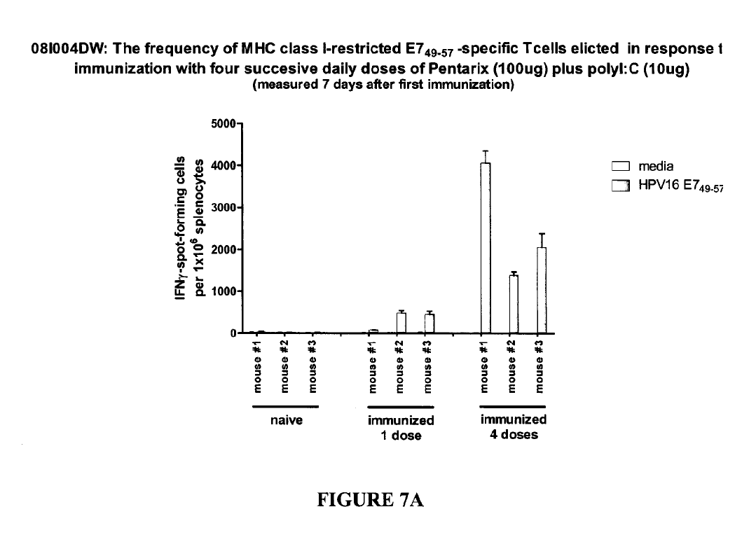

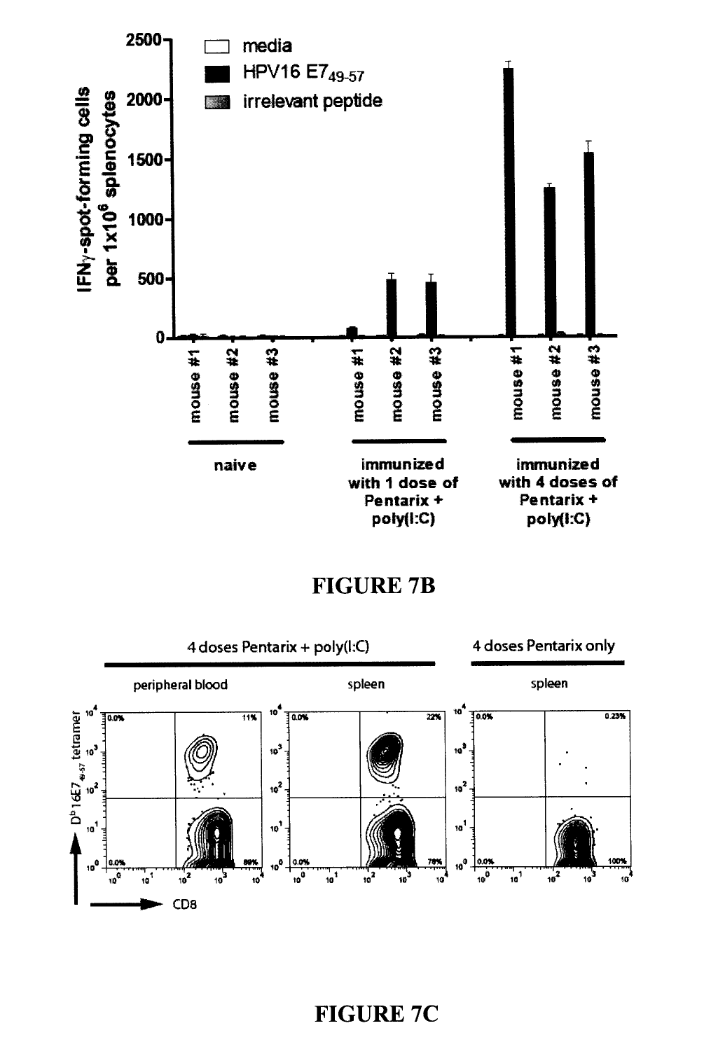

In another study (FIG. 7A), mice (naïve C57B1/6) were left untreated or were immunized subcutaneously one time or 4 times (daily on days 1-4) with 100 μg of recombinant Pentarix protein admixed with 10 μg of the TLR3 agonist polyI-C (Amersham or Sigma). Mice receiving multiple consecutive daily immunizations were immunized at approximately 24 hour intervals. Seven days after the initial immunization mice were euthanized and spleens were excised. Single cell suspensions of splenocytes were prepared in 10 ml of cRPMI (RPMI 1640, 10% FCS, 2 mM L-glutamine, 50 uM 2-mercaptoethanol, 10 mM HEPES and 10 mM sodium pyruvate) by mashing spleens through a 70 uM filter using the plunger from a 5 ml syringe. ELISPOT plates (MSIP, Millipore) were pre-coated overnight with 10 μg/ml anti-IFN-γ capture antibody (AN18-Mabtech) and then blocked for 2 hours at 37° C. with cRPMI. Splenocytes (3×10⁵ cells per well) were plated in triplicate in the absence of any stimulus (media only), or in the presence of 10 μg/ml E7₄₉₋₅₇ peptide. After overnight incubation at 37° C., ELISPOT plates were washed and incubated for 2 hours at 37° C. with 1 μg/ml biotinylated anti-mouse IFN-γ (mAb R4-6A2, Mabtech) followed by development with Vectastain ABC Elite kit and Vectastain AEC substrate reagent according to manufacturers' instructions (Vector Labs). Spots were quantitated using a commercial ELISPOT counting service (Zellnet). Results are presented as the number of IFN-γ spot forming cells per 1×10⁶ splenocytes when cultured in the presence of media only or media plus HPV 16 E7₄₉₋₅₇ peptide.

In another study, we found that in mice receiving 4 successive doses of Pentarix protein plus poly(I:C), up to 11% of CD8 T cells in peripheral blood and up to 22% of CD8 T cells in the spleen stained positively with H-2D^{b} HPV 16 E7₄₉₋₅₇ tetramer (FIG. 7C). In this study, HPV-specific T cells were not detectable in the spleens of mice immunized with 4 doses of Pentarix protein only, indicating the importance of adjuvant for CD8 T cell expansion under the study conditions.

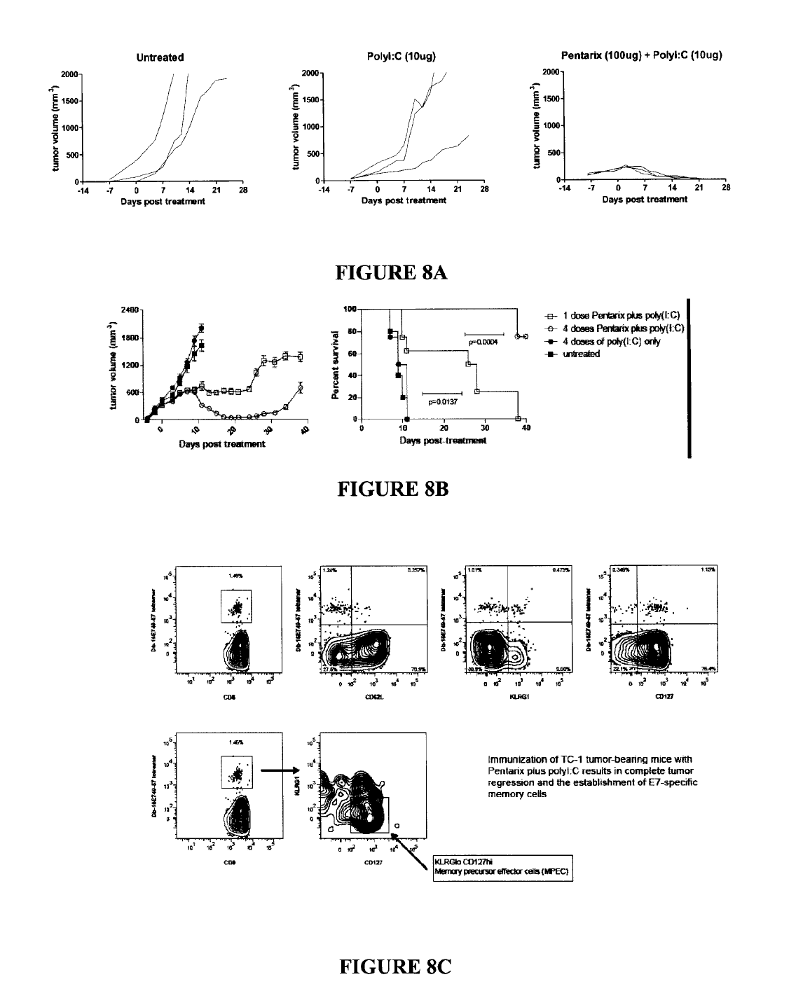

In another study (FIG. 8A), naïve C57B1/6 mice (3 mice per cohort) were implanted with E7-expressing TC 1 tumors cells (1×10⁵ per mouse), subcutaneously into the left flank, on day −14. On day 0 (when tumors reached approximately 200 mm³ in size) mice were either left untreated, or were treated with a single inoculation (subcutaneous in the scruff of the neck) of polyI-C only (10 μg) or Pentarix protein (100 μg) plus the TLR3 agonist polyI-C (10 μg). Mice immunized with Pentarix protein (100 μg) plus the TLR3 agonist polyI-C (10 μg) (FIG. 8A, right panel) fully regressed tumors and remained tumor free for the duration of the study (60 days). Tumors were measured every 2-3 days using an electronic digital caliper and volumes were calculated using the formula width²×length×0.5 and tumor-bearing mice were euthanized when the tumor volume reached approximately 2000 mm³ according to the CCAC (Canadian Council on Animal Care) guidelines.

In another study, TC-1 tumor cells were grown in cRPMI containing 0.4 mg/ml G418 to 60-80% confluency and were harvested by a brief exposure to 0.25% trypsin followed by neutralization with cRPMI. TC-1 tumor cells (1×10⁵ per mouse) were implanted subcutaneously into the left flank of naïve C57B1/6 mice and tumor growth was monitored by measuring the tumor every two to three days using electronic calipers. Tumor volumes were calculated using the formula width²×length×0.5. Tumor-bearing mice were euthanized when the tumor volume exceeded 2000 mm³ according to the CCAC (Canadian Council on Animal Care) guidelines.

In this study, the increased level of E7-specific CD8 T cells evoked by cluster vaccination also conferred an improved ability to regress E7-expresing TC 1 tumors, which was demonstrated when tumors were allowed to grow to a larger size than normal prior to the initiation of treatment (FIG. 8B). Mice that harbored tumors with an average volume of 350 mm³ at time of treatment were immunized with either a single dose or 4 successive daily doses of Pentarix plus poly(I:C). Five of 8 mice receiving a single dose of vaccine exhibited transient (but incomplete) tumor regression and significantly improved time of survival compared to untreated mice or mice treated with poly(I:C) only. However, all mice receiving a single dose of vaccine eventually succumbed to progressive tumor growth. In contrast, of mice that received 4 successive doses of Pentarix plus poly(I:C), 100% (8 of 8) fully regressed these large tumors to the point that they were no longer palpable. Although some tumors began to recur 4-5 weeks after treatment, 75% of mice (6 of 8) in the 4-dose cohort were still alive at day 38 and 50% remained tumor free. All mice in all other cohorts had been euthanized due to progressive tumor growth by this time point.

In another study, in which naïve C57B1/6 mice were implanted with E7-expressing TC 1 tumors cells, immunization of TC 1-tumor bearing mice with Pentarix protein to plus polyI:C elicited complete tumor regression and the establishment of E7-specific CD8 memory cells that persisted after tumor regression (FIG. 8C).

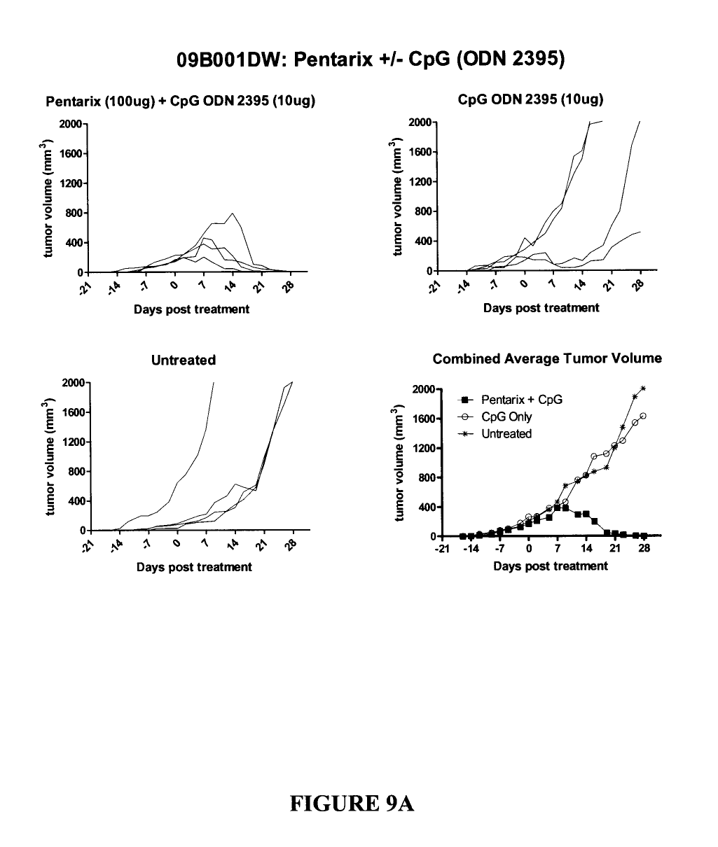

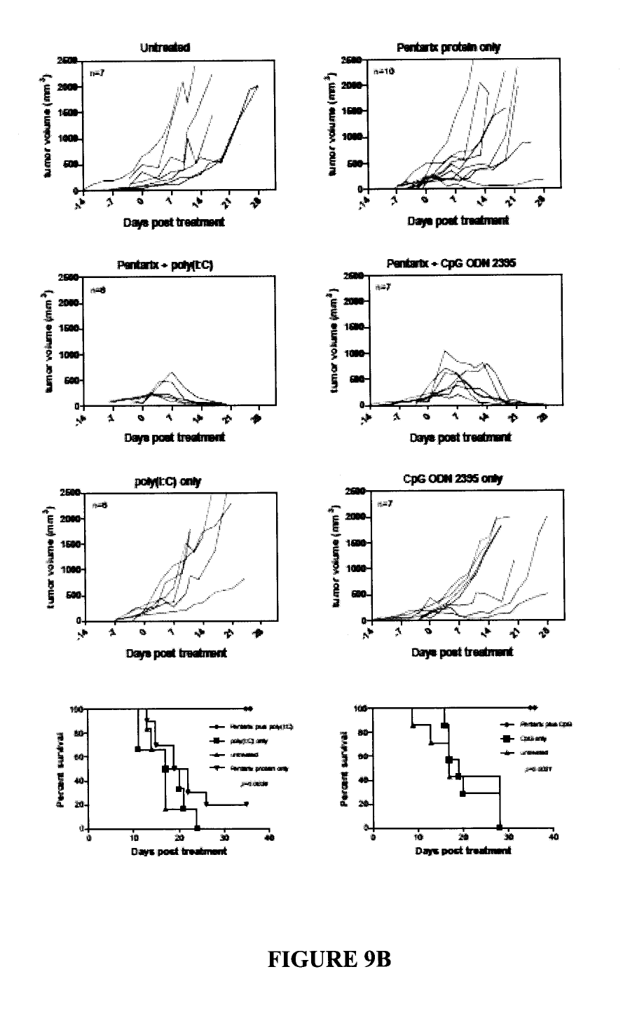

In another study (FIG. 9A), HPV E7-expressing TC-1 tumor cells (1×10⁵ cells per mouse) were implanted subcutaneously into the left flank of naïve C57B1/6 mice (4 mice per cohort) on day −21. On day 0 (when tumors reached approximately 200 mm³ in size) mice were treated with a single inoculation (subcutaneous in the scruff of the neck) of Pentarix protein (100 μg) plus 10 μg of the TLR9 agonist CpG 2395 (Invivogen) or 10 μg of the TLR9 agonist CpG 2395 only (no Pentarix) or were left untreated. Mice immunized with Pentarix protein (100 μg) plus 10 μg of the TLR9 agonist CpG 2395 fully regressed tumors and remained tumor free for the duration of the study. Tumor growth was monitored by measuring the tumor every two to three days using calipers and tumor volumes were calculated using the formula width²×length×0.5 and tumor-bearing mice were euthanized when the tumor volume reached approximately 2000 mm³ according to the CCAC (Canadian Council on Animal Care) guidelines.

In another study to assess the effector function of CD8 T cells elicited by a single immunization with Pentarix protein plus these adjuvants, in terms of their ability to regress established, E7-expressing tumors, HPV16 E7-expressing TC-1 tumors were implanted subcutaneously in naïve recipient mice and were allowed to grow until they reached a volume of approximately 200 mm³. Animals immunized subcutaneously with Pentarix protein admixed with either poly(I:C) or CpG oligonucleotide began to regress these tumors, generally within one week of immunization (FIG. 9B). In this study, all animals immunized with Pentarix protein plus adjuvant had complete tumor regression by three weeks post-immunization and remained tumor free for at least three months. In contrast, mice that were either untreated or that were treated with adjuvant only or Pentarix protein only displayed progressive tumor growth and were euthanized (generally within 28 days of tumor implantation) due to excessive tumor burden.

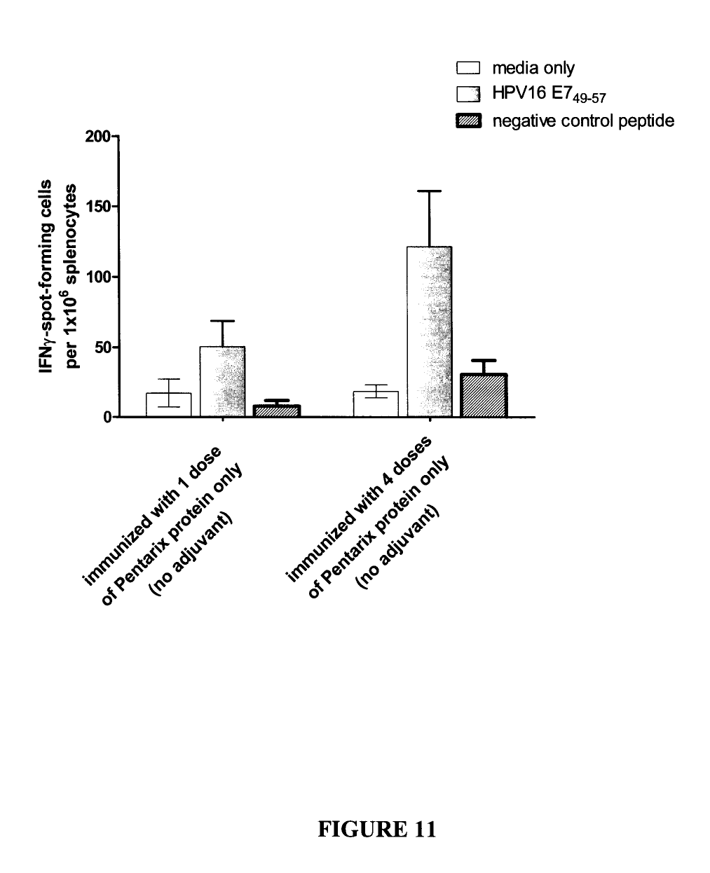

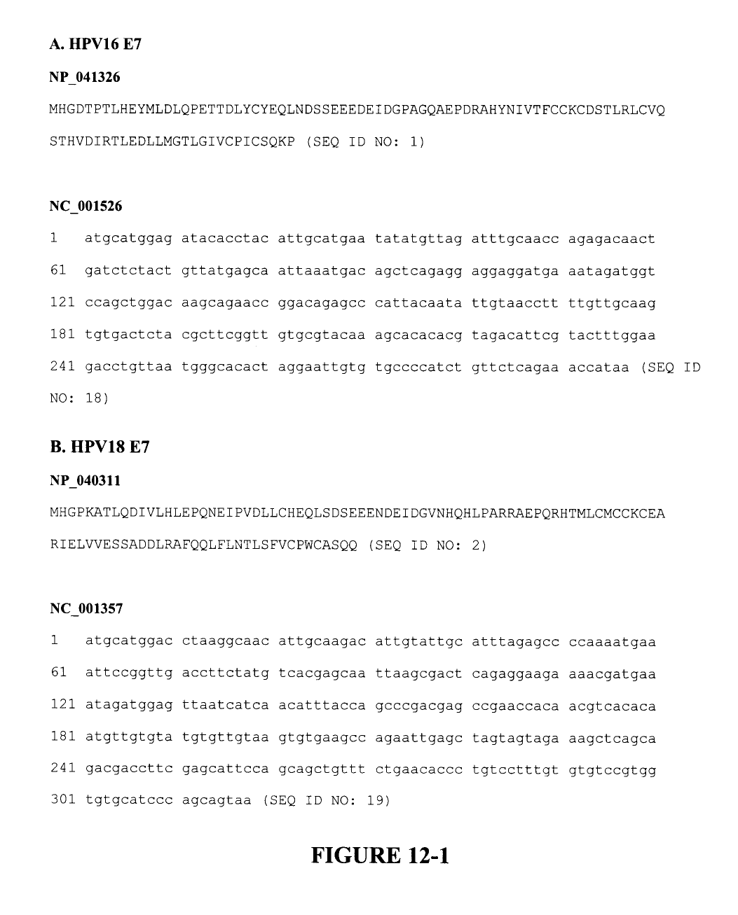

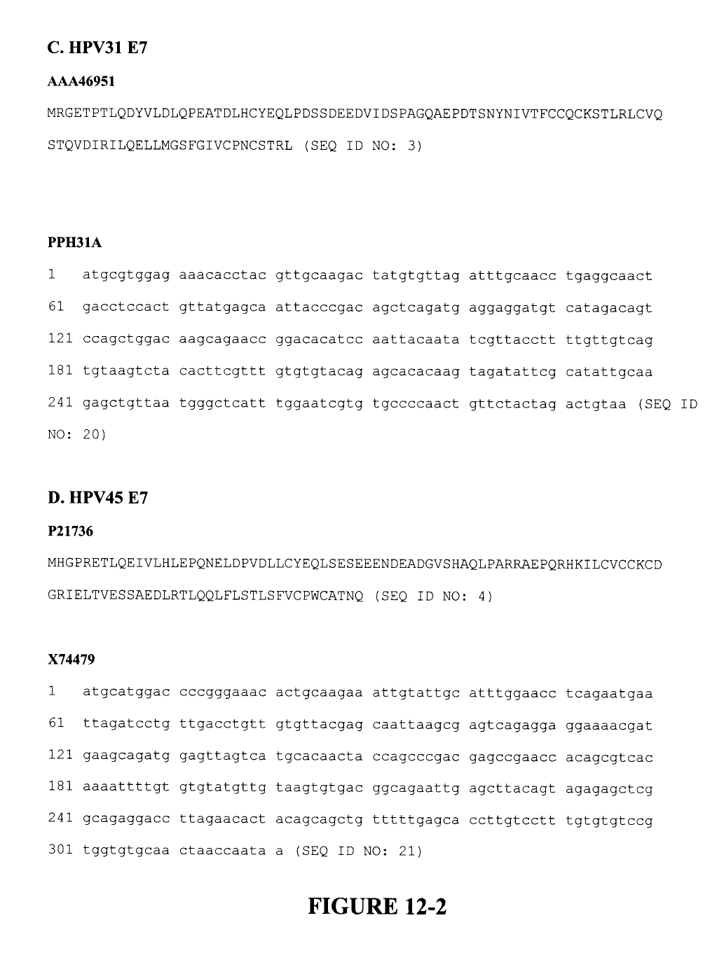

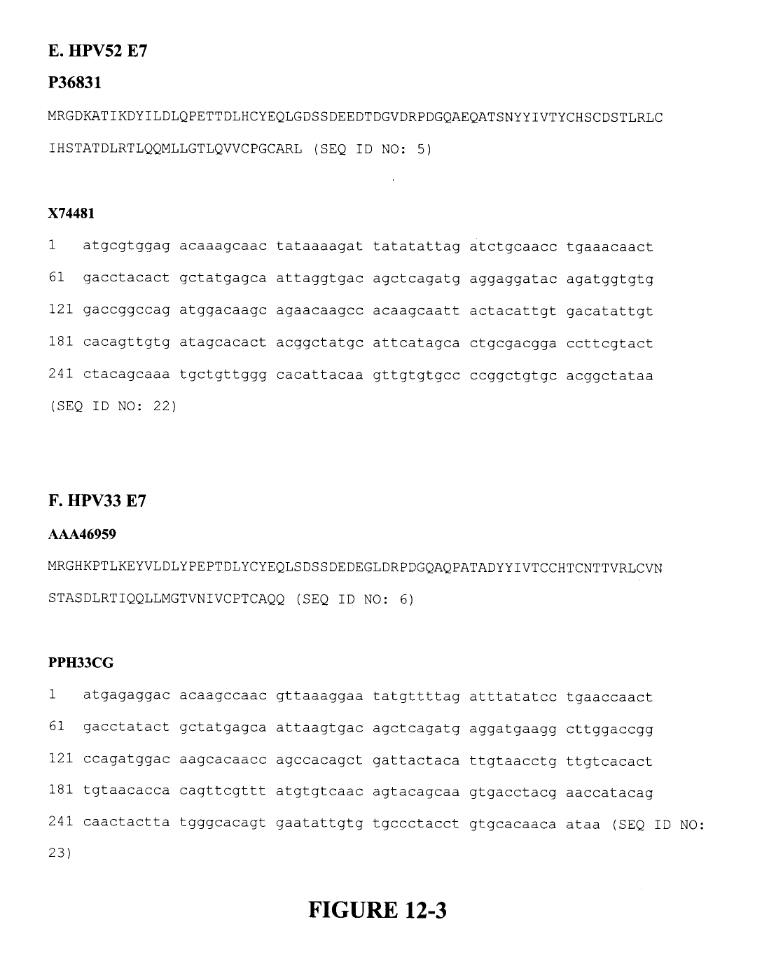

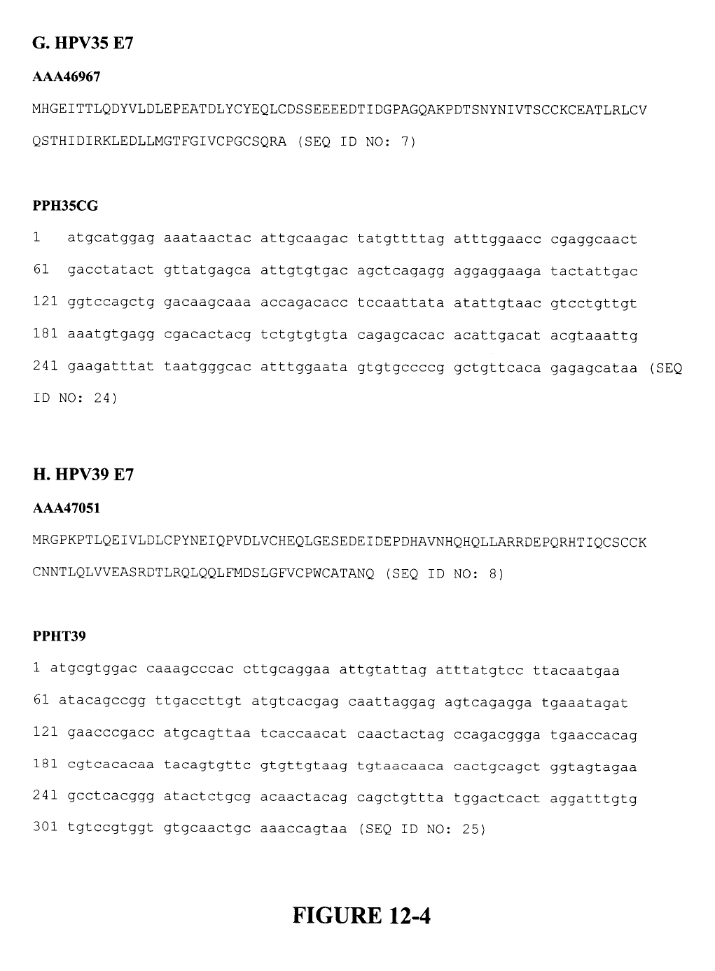

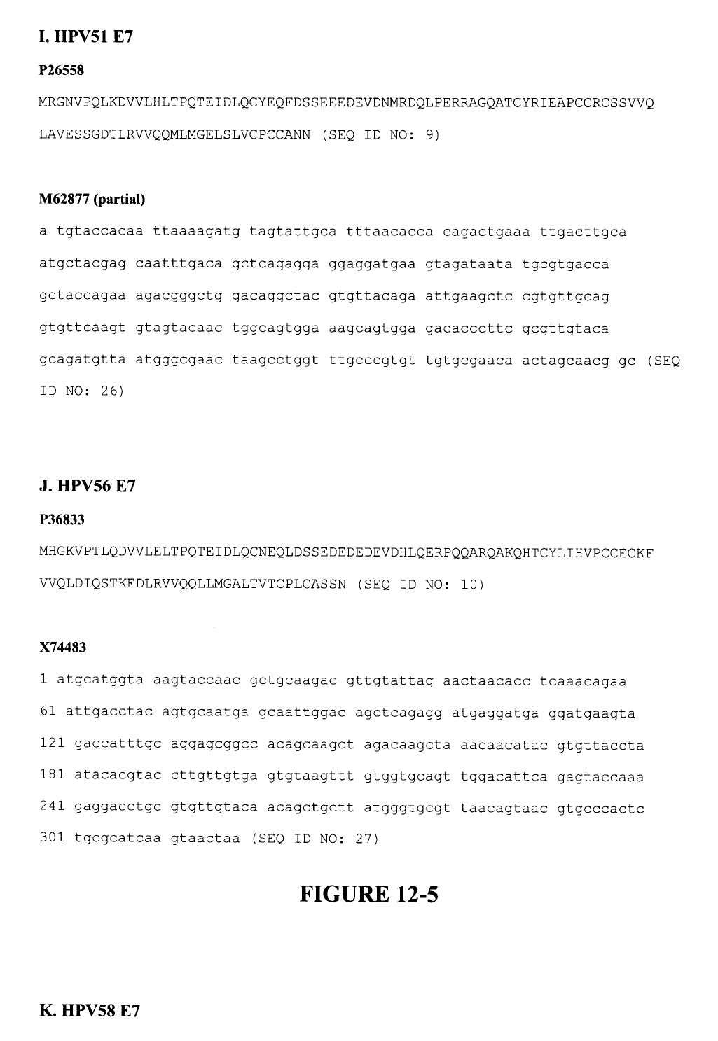

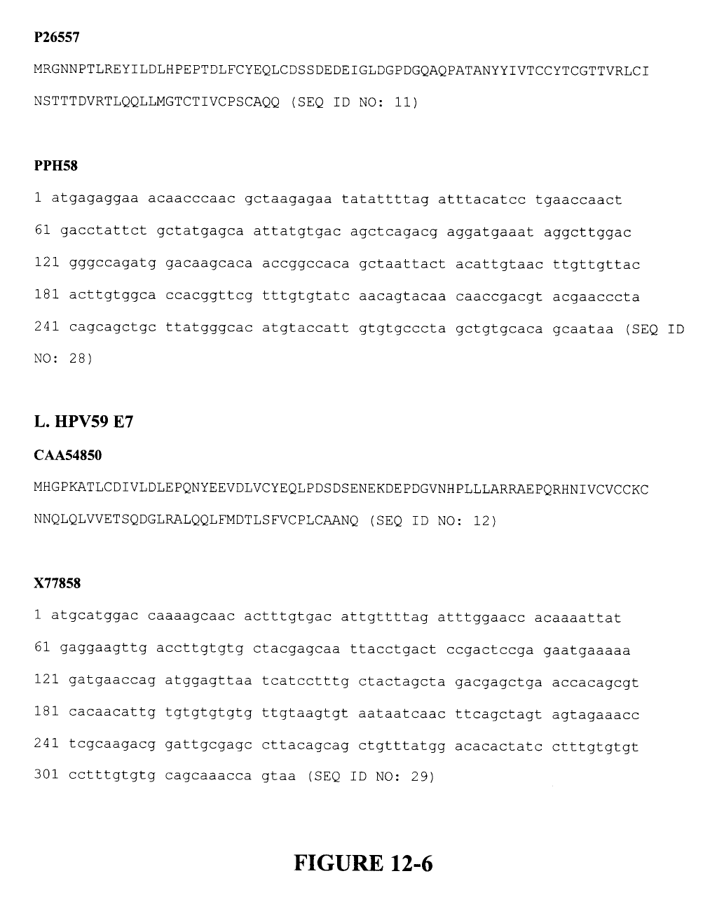

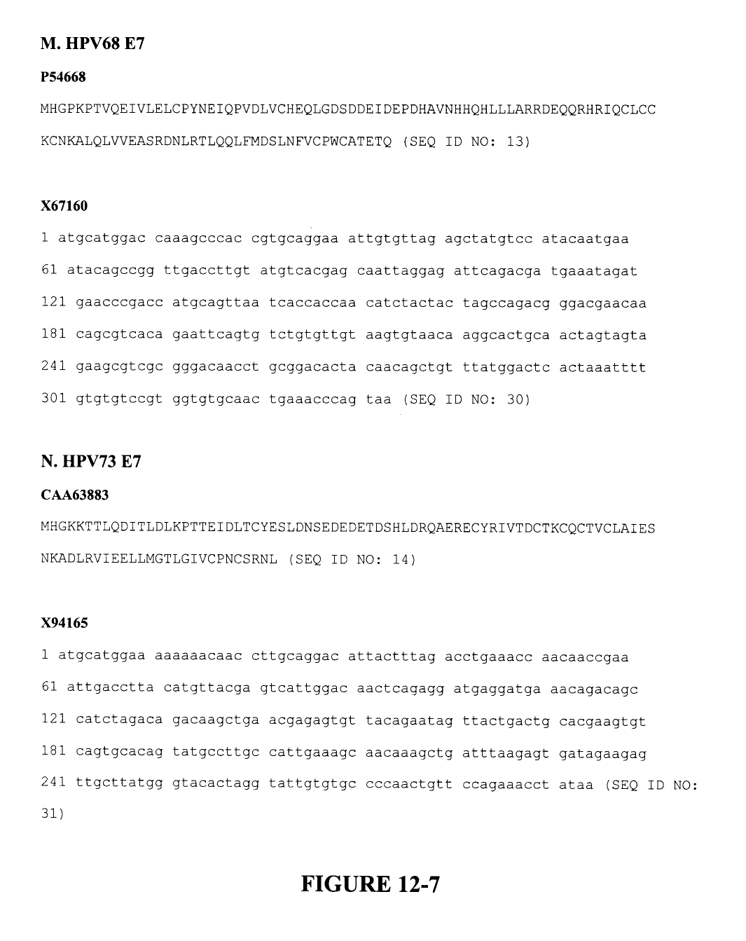

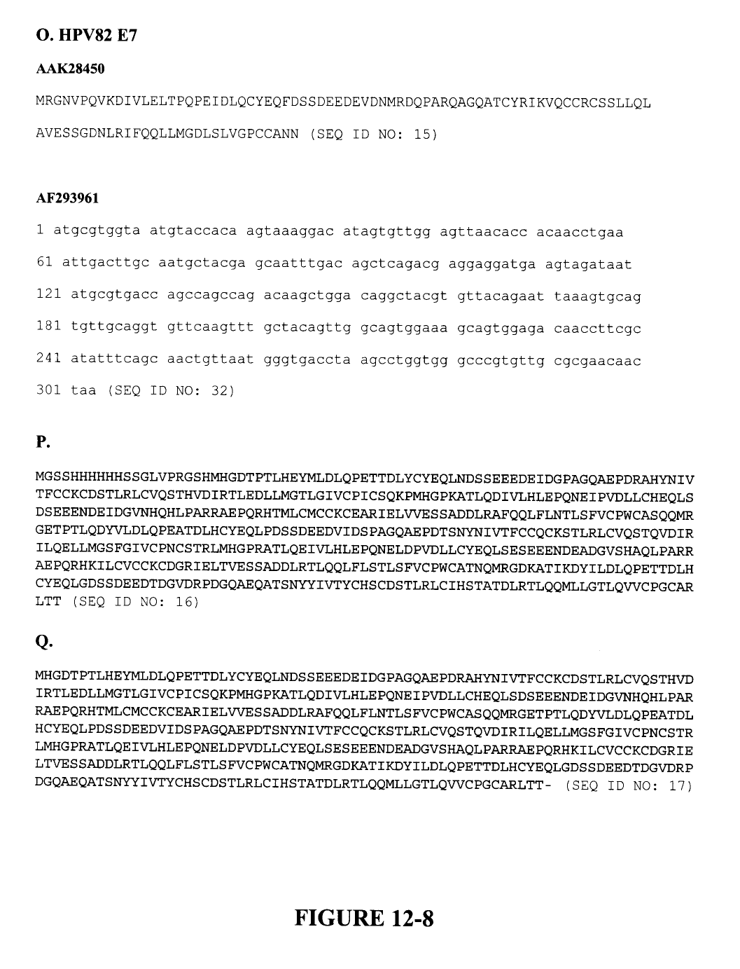

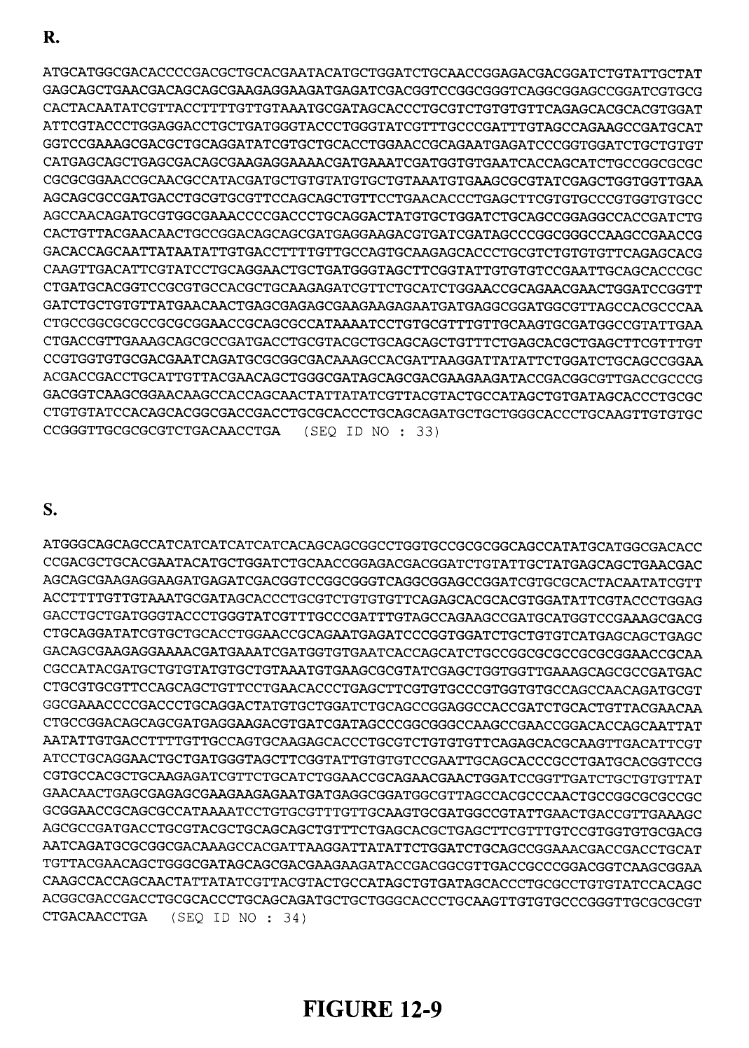

In summary, as was observed with the OVA protein, Pentarix elicited strong CD8+ T-cell mediated immune responses when admixed with agonists of either TLR3 (FIGS. 5A and 5B) or TLR9 (FIGS. 6A and 6B). Also as was observed with the model antigen OVA, application of the sequential daily (cluster) immunization strategy substantially enhanced the CD8+ immune response elicited by Pentarix (FIGS. 7A-C). Pentarix also elicited CD8+ T-cell mediated immune responses without adjuvant (FIG. 11).

Immune responses elicited by vaccination with Pentarix protein were capable of regressing established TC-1 tumors within days of vaccination (FIGS. 8A, 8B, 9A and 9B). This was true when Pentarix was combined with either poly(I:C) (FIGS. 8A, 8B and 9B) or CpG oligonucleotide (FIGS. 9A and 9B). In two different experiments, complete or near complete regression of established TC-1 tumors was achieved in all mice receiving Pentarix whereas 100% of control mice (no treatment or adjuvant only) succumbed to progressively growing tumors.

### Example 5

### Pentarix Elicits Immunity to a HPV31 E7 Epitope

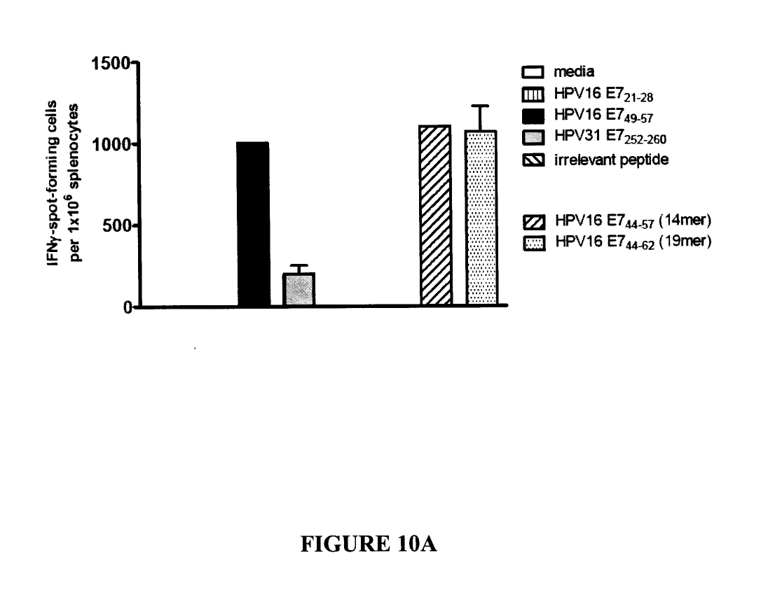

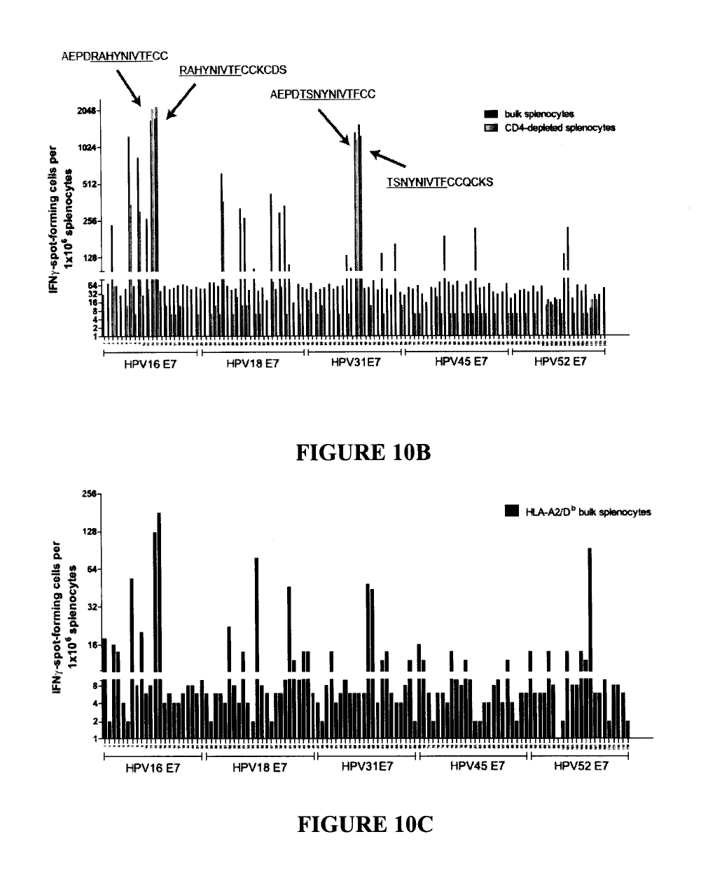

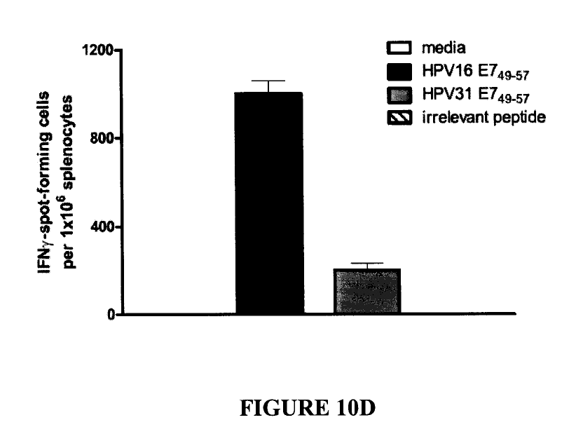

Naïve C57B1/6 mice were immunized with 100 μg of whole, soluble Pentarix protein admixed with 10 μg of poly(I:C) (Amersham). Seven days post-immunization mice were euthanized and bulk splenocyte preparations were assessed by IFN-γ ELISPOT to quantitate the number of CD8+ T cells specific for peptides identified using two different predictive MHC-binding algorithms (SYFPEITHI and IEDB). Results are reported as the number of IFN-γ spot-forming cells per 1×10⁶ splenocytes after stimulation with media only or the indicated peptide (10 μg/ml). Both the long (14mer and 20mer) and short (9mer) versions of the HPV16 E7₄₉₋₅₇ peptide were found to elicit responses from immunized mice, as well as the HPV31-derived candidate peptide (HPV31 E7₂₅₂₋₂₆₀-TSNYNIVTF; SEQ ID NO: 35) with the highest H2 Db score on both algorithms (FIG. 10A). This finding represents a new epitope for HPV31 E7 and demonstrates that Pentarix elicits immunity to a HPV31 E7 epitope.

### Example 6

### Pentarix Elicits Immune Responses Against Multiple Genotypes of HPV

To assess the scope of the cellular immune response elicited by Pentarix, bulk splenocytes and CD4-depleted splenocytes from mice immunized with Pentarix plus poly(I:C) were analyzed directly ex vivo by ELISPOT with a library of overlapping 15mer peptides that spanned the entire Pentarix protein sequence. Where indicated, CD4 cells were depleted from bulk splenocytes using magnetic depletion. Briefly, bulk splenocytes were stained with PE-conjugated anti-CD4 antibody (clone L3T4; BD Biosciences) and labeled cells were depleted using anti-PE microbeads according to manufacturer's instructions (Miltenyi). As shown in FIG. 10B, the response elicited in C57B1/6 mice by Pentarix encompassed all five of the HPV strains contained within the vaccine. Peptides containing the well-characterized H-2D^{b}-restricted epitope HPV16 E7₄₉₋₅₇ (RAHYNIVTF; SEQ ID NO: 38) comprised the strongest response in terms of absolute numbers of antigen-specific CD8 T cells. The next strongest response was elicited by 15mer peptides from HPV31 encompassing a related peptide (TSNYNIVTF; SEQ ID NO: 35) that is predicted by algorithm analyses to be an even stronger binder to H-2D^{b} than HPV16 E7₄₉₋₅₇. A variety of 15mer peptides from other HPV E7 protein sequences also elicited responses of varying intensity from both bulk and CD4-depleted splenocytes, confirming that Pentarix is capable of eliciting a broad scope cellular immune response, even in inbred mice with a limited repertoire of MHC molecules. In addition, HLA-A2 transgenic mice (HLA-A2/D^{b}, Jackson Labs stock #004191) were also immunized with Pentarix plus poly(I:C) and assessed by ELISPOT using the same library of overlapping 15mer peptides. Interestingly, although the general strength of the response was greater in C57B1/6 than HLA-A2/D^{b} mice, the overall complexity of the response was very similar (FIG. 10C), suggesting that an H2^{b}-restricted response had been elicited but that an HLA-A2 restricted response had not. Furthermore, as has been observed in a number of other studies using HPV 16 E7 antigen, we were unable to detect a response against the HLA-A2 restricted 11-20 or 86-93 minimal peptide epitopes of HPV16 E7 in mice immunized with Pentarix plus poly(I:C).

To confirm that TSNYNIVTF (SEQ ID NO: 35) was the precise minimal epitope within the strongly reactive HPV31 15mer peptides AEPDTSNYNIVTFCC (SEQ ID NO: 36) and TSNYNIVTFCCQCKS (SEQ ID NO: 37) splenocytes from mice immunized with Pentarix plus poly(I:C) were assessed by ELISPOT and were found to be responsive to this 9mer minimal peptide (FIG. 10D).

### Other Embodiments

Although various embodiments of the invention are disclosed herein, many adaptations and modifications may be made within the spirit and scope of the invention in accordance with the common general knowledge of those skilled in this art. Such modifications include the substitution of known equivalents for any aspect of the invention in order to achieve the same result in substantially the same way. Accession numbers, as used herein, may refer to Accession numbers from multiple databases, including GenBank, the European Molecular Biology Laboratory (EMBL), the DNA Database of Japan (DDBJ), or the Genome Sequence Data Base (GSDB), for nucleotide sequences, and including the Protein Information Resource (PIR), SWISSPROT, Protein Research Foundation (PRF), and Protein Data Bank (PDB) (sequences from solved structures), as well as from translations from annotated coding regions from nucleotide sequences in GenBank, EMBL, DDBJ, or RefSeq, for polypeptide sequences. Numeric ranges are inclusive of the numbers defining the range, and of sub-ranges encompassed therein. As used herein, the terms “comprising”, “comprises”, “having” or “has” are used as an open-ended terms, substantially equivalent to the phrase “including, but not limited to”. Terms such as “the,” “a,” and “an” are to be construed as indicating either the singular or plural. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

The present invention has been described with regard to one or more embodiments. However, it will be apparent to persons skilled in the art that a number of variations and modifications can be made without departing from the scope of the invention as defined in the claims.

### REFERENCES

1. Schiffman, M., P. E. Castle, J. Jeronimo, A. C. Rodriguez, and S. Wacholder. 2007. Human papillomavirus and cervical cancer. Lancet 370:890-907.
2. Bosch, F. X., A. Lorincz, N. Munoz, C. J. Meijer, and K. V. Shah. 2002. The causal relation between human papillomavirus and cervical cancer. Journal of clinical pathology 55:244-265.
3. Castellsague, X. 2008. Natural history and epidemiology of HPV infection and cervical cancer. Gynecol Oncol 110:S4-7.
4. Psyrri, A., and D. DiMaio. 2008. Human papillomavirus in cervical and head-and-neck cancer. Nature clinical practice. Oncology 5:24-31.
5. Devaraj, K., M. L. Gillison, and T. C. Wu. 2003. Development of HPV vaccines for HPV-associated head and neck squamous cell carcinoma. Critical reviews in oral biology and medicine: an official publication of the American Association of Oral Biologists 14:345-362.
6. Syrjanen, S. 2005. Human papillomavirus (HPV) in head and neck cancer. J Clin Viral 32 Suppl 1:S59-66.
7. Winer, R. L., S. K. Lee, J. P. Hughes, D. E. Adam, N. B. Kiviat, and L. A. Koutsky. 2003. Genital human papillomavirus infection: incidence and risk factors in a cohort of female university students. American Journal of Epidemiology 157:218-226.
8. Frazer, I. H.2004. Prevention of cervical cancer through papillomavirus vaccination. Nature reviews. Immunology 4:46-54.
9. Cutts, F. T., S. Franceschi, S. Goldie, X. Castellsague, S. de Sanjose, G. Garnett, W. J. Edmunds, P. Claeys, K. L. Goldenthal, D. M. Harper, and L. Markowitz. 2007. Human papillomavirus and HPV vaccines: a review. Bull World Health Organ 85:719-726.
10. Leggatt, G. R., and I. H. Frazer. 2007. HPV vaccines: the beginning of the end for cervical cancer. Curr Opin Immunol 19:232-238.
11. Munoz, N., F. X. Bosch, S. de Sanjose, R. Herrero, X. Castellsague, K. V. Shah, P. J. Snijders, C. J. Meijer, and G. International Agency for Research on Cancer Multicenter Cervical Cancer Study. 2003. Epidemiologic classification of human papillomavirus types associated with cervical cancer. The New England journal of medicine 348:518-527.
12. Bosch, F. X., M. M. Manos, N. Munoz, M. Sherman, A. M. Jansen, J. Peto, M. H. Schiffman, V. Moreno, R. Kurman, and K. V. Shah. 1995. Prevalence of human papillomavirus in cervical cancer: a worldwide perspective. International biological study on cervical cancer (IBSCC) Study Group. Journal of the National Cancer Institute 87:796-802.
13. Jenkins, D. 2008. A review of cross-protection against oncogenic HPV by an HPV-16/18 ASO₄-adjuvanted cervical cancer vaccine: importance of virological and clinical endpoints and implications for mass vaccination in cervical cancer prevention. Gynecol Oncol 110:S18-25.
14. Ault, K. A. 2007. Human papillomavirus vaccines and the potential for cross-protection between related HPV types. Gynecol Oncol 107:S31-33.
15. Harper, D. M., E. L. Franco, C. M. Wheeler, A. B. Moscicki, B. Romanowski, C. M. Roteli-Martins, D. Jenkins, A. Schuind, S. A. Costa Clemens, and G. Dubin. 2006. Sustained efficacy up to 4.5 years of a bivalent L1 virus-like particle vaccine against human papillomavirus types 16 and 18: follow-up from a randomised control trial. Lancet 367:1247-1255.
16. Hildesheim, A., R. Herrero, S. Wacholder, A. C. Rodriguez, D. Solomon, M. C. Bratti, J. T. Schiller, P. Gonzalez, G. Dubin, C. Porras, S. E. Jimenez, and D. R. Lowy. 2007. Effect of human papillomavirus 16/18 L1 viruslike particle vaccine among young women with preexisting infection: a randomized trial. Jama 298:743-753.
17. Herzog, T. J., W. K. Huh, L. S. Downs, J. S. Smith, and B. J. Monk. 2008. Initial lessons learned in HPV vaccination. Gynecol Oncol 109:S4-11.
18. Goldie, S. J., M. O'Shea, M. Diaz, and S. Y. Kim. 2008. Benefits, cost requirements and cost-effectiveness of the HPV16,18 vaccine for cervical cancer prevention in developing countries: policy implications. Reprod Health Matters 16:86-96.
19. Monie, A., C. F. Hung, and T. C. Wu. 2007. Preventive and therapeutic HPV vaccines. Curr Opin Investig Drugs 8:1038-1050.
20. Hung, C. F., B. Ma, A. Monie, S. W. Tsen, and T. C. Wu. 2008. Therapeutic human papillomavirus vaccines: current clinical trials and future directions. Expert Opin Biol Ther 8:421-439.
21. Narisawa-Saito, M., and T. Kiyono. 2007. Basic mechanisms of high-risk human papillomavirus-induced carcinogenesis: roles of E6 and E7 proteins. Cancer science 98:1505-1511.
22. Crook, T., J. P. Morgenstern, L. Crawford, and L. Banks. 1989. Continued expression of HPV-16 E7 protein is required for maintenance of the transformed phenotype of cells co-transformed by HPV-16 plus EJ-ras. Embo J 8:513-519.
23. Smotkin, D., and F. O. Wettstein. 1987. The major human papillomavirus protein in cervical cancers is a cytoplasmic phosphoprotein. J Virol 61:1686-1689.
24. Munger, K., A. Baldwin, K. M. Edwards, H. Hayakawa, C. L. Nguyen, M. Owens, M. Grace, and K. Huh. 2004. Mechanisms of human papillomavirus-induced oncogenesis. J Virol 78:11451-11460.
25. Lin, K. Y., F. G. Guarnieri, K. F. Staveley-O'Carroll, H. I. Levitsky, J. T. August, D. M. Pardoll, and T. C. Wu. 1996. Treatment of established tumors with a novel vaccine that enhances major histocompatibility class II presentation of tumor antigen. Cancer research 56:21-26.
26. Feltkamp, M. C., H. L. Smits, M. P. Vierboom, R. P. Minnaar, B. M. de Jongh, J. W. Drijfhout, J. ter Schegget, C. J. Melief, and W. M. Kast. 1993. Vaccination with cytotoxic T lymphocyte epitope-containing peptide protects against a tumor induced by human papillomavirus type 16-transformed cells. European journal of immunology 23:2242-2249.
27. Vierboom, M. P., M. C. Feltkamp, A. Neisig, J. W. Drijfhout, J. ter Schegget, J. J. Neefjes, C. J. Melief, and W. M. Kast. 1998. Peptide vaccination with an anchor-replaced CTL epitope protects against human papillomavirus type 16-induced tumors expressing the wild-type epitope. Journal of immunotherapy (Hagerstown, Md.: 1997) 21:399-408.
28. Manuri, P. R., B. Nehete, P. N. Nehete, R. Reisenauer, S. Wardell, A. N. Courtney, R. Gambhira, D. Lomada, A. K. Chopra, and K. J. Sastry. 2007. Intranasal immunization with synthetic peptides corresponding to the E6 and E7 oncoproteins of human papillomavirus type 16 induces systemic and mucosal cellular immune responses and tumor protection. Vaccine 25:3302-3310.
29. Chen, W., W. Yan, and L. Huang. 2008. A simple but effective cancer vaccine consisting of an antigen and a cationic lipid. Cancer immunology, immunotherapy: CII 57:517-530.
30. Berraondo, P., C. Nouze, X. Preville, D. Ladant, and C. Leclerc. 2007. Eradication of large tumors in mice by a tritherapy targeting the innate, adaptive, and regulatory components of the immune system. Cancer research 67:8847-8855.
31. Peng, S., H. Ji, C. Trimble, L. He, Y. C. Tsai, J. Yeatermeyer, D. A. Boyd, C. F. Hung, and T. C. Wu. 2004. Development of a DNA vaccine targeting human papillomavirus type 16 oncoprotein E6. Journal of virology 78:8468-8476.
32. Lin, C. T., Y. C. Tsai, L. He, R. Calizo, H. H. Chou, T. C. Chang, Y. K. Soong, C. F. Hung, and C. H. Lai. 2006. A DNA vaccine encoding a codon-optimized human papillomavirus type 16 E6 gene enhances CTL response and anti-tumor activity. Journal of Biomedical Science 13:481-488.
33. Cheng, W. F., C. F. Hung, S. I. Pai, K. F. Hsu, L. He, M. Ling, and T. C. Wu. 2002. Repeated DNA vaccinations elicited qualitatively different cytotoxic T lymphocytes and improved protective antitumor effects. Journal of Biomedical Science 9:675-687.
34. Borysiewicz, L. K., A. Fiander, M. Nimako, S. Man, G. W. Wilkinson, D. Westmoreland, A. S. Evans, M. Adams, S, N. Stacey, M. E. Boursnell, E. Rutherford, J. K. Hickling, and S. C. Inglis. 1996. A recombinant vaccinia virus encoding human papillomavirus types 16 and 18, E6 and E7 proteins as immunotherapy for cervical cancer. Lancet 347:1523-1527.
35. L1, J., Y. Sun, and A. Garen. 2002. Immunization and immunotherapy for cancers involving infection by a human papillomavirus in a mouse model. Proceedings of the National Academy of Sciences of the United States of America 99:16232-16236.
36. Zhang, L., Y. Tang, H. Akbulut, D. Zelterman, P J Linton, and A. B. Deisseroth. 2003. An adenoviral vector cancer vaccine that delivers a tumor-associated antigen/CD40-ligand fusion protein to dendritic cells. Proceedings of the National Academy of Sciences of the United States of America 100:15101-15106.
37. Jin, H. S., E. K. Park, J. M. Lee, S. E. NamKoong, D. G. Kim, Y. J. Lee, H. J. Jun, B. D. Han, S. M. Bae, and W. S. Ahn. 2005. Immunization with adenoviral vectors carrying recombinant IL-12 and E7 enhanced the antitumor immunity to human papillomavirus 16-associated tumor. Gynecologic oncology 97:559-567.
38. Londono, L. P., S. Chatfield, R. W. Tindle, K. Herd, X. M. Gao, I. Frazer, and G. Dougan. 1996. Immunisation of mice using Salmonella typhimurium expressing human papillomavirus type 16 E7 epitopes inserted into hepatitis B virus core antigen. Vaccine 14:545-552.
39. Krul, M. R., E. J. Tijhaar, J. A. Kleijne, A. M. Van Loon, M. G. Nievers, H. Schipper, L. Geerse, M. Van der Kolk, P. A. Steerenberg, F. R. Mooi, and W. Den Otter. 1996. Induction of an antibody response in mice against human papillomavirus (HPV) type 16 after immunization with HPV recombinant Salmonella strains. Cancer immunology, immunotherapy: CII 43:44-48.
40. Gunn, G. R., A. Zubair, C. Peters, Z. K. Pan, T. C. Wu, and Y. Paterson. 2001. Two Listeria monocytogenes vaccine vectors that express different molecular forms of human papilloma virus-16 (HPV-16) E7 induce qualitatively different T cell immunity that correlates with their ability to induce regression of established tumors immortalized by HPV-16. Journal of immunology (Baltimore, Md.: 1950) 167:6471-6479.
41. Lin, C. W., J. Y. Lee, Y. P. Tsao, C. P. Shen, H. C. Lai, and S. L. Chen. 2002. Oral vaccination with recombinant Listeria monocytogenes expressing human papillomavirus type 16 E7 can cause tumor growth in mice to regress. International journal of cancer. Journal international du cancer 102:629-637.
42. Mackova, J., L. Kutinova, P. Hainz, J. Krystofova, V. Sroller, P. Otahal, P. Gabriel, and S, Nemeckova. 2004. Adjuvant effect of dendritic cells transduced with recombinant vaccinia virus expressing HPV 16-E7 is inhibited by co-expression of IL12. International journal of oncology 24:1581-1588.
43. Hauser, H., L. Shen, Q. L. Gu, S. Krueger, and S. Y. Chen. 2004. Secretory heat-shock protein as a dendritic cell-targeting molecule: a new strategy to enhance the potency of genetic vaccines. Gene therapy 11:924-932.
44. Kang, T. H., J. H. Lee, H. C. Bae, K. H. Noh, J. H. Kim, C. K. Song, B. C. Shin, C. F. Hung, T. C. Wu, J. S. Park, and T. W. Kim. 2006. Enhancement of dendritic cell-based vaccine potency by targeting antigen to endosomal/lysosomal compartments. Immunology letters 106:126-134.
45. Chandy, A. G., M. Nurkkala, A. Josefsson, and K. Eriksson. 2007. Therapeutic dendritic cell vaccination with Ag coupled to cholera toxin in combination with intratumoural CpG injection leads to complete tumour eradication in mice bearing HPV 16 expressing tumours. Vaccine 25:6037-6046.
46. Greenstone, H. L., J. D. Nieland, K. E. de Visser, M. L. De Bruijn, R. Kirnbauer, R. B. Roden, D. R. Lowy, W. M. Kast, and J. T. Schiller. 1998. Chimeric papillomavirus virus-like particles elicit antitumor immunity against the E7 oncoprotein in an HPV 16 tumor model. Proceedings of the National Academy of Sciences of the United States of America 95:1800-1805.
47. Da Silva, D. M., J. T. Schiller, and W. M. Kast. 2003. Heterologous boosting increases immunogenicity of chimeric papillomavirus virus-like particle vaccines. Vaccine 21:3219-3227.
48. Warrino, D. E., W. C. Olson, M. I. Scarrow, L. J. D'Ambrosio-Brennan, R. S. Guido, D. M. Da Silva, W. M. Kast, and W. J. Storkus. 2005. Human papillomavirus L1L2-E7 virus-like particles partially mature human dendritic cells and elicit E7-specific T-helper responses from patients with cervical intraepithelial neoplasia or cervical cancer in vitro. Human immunology 66:762-772.
49. Kaufmann, A. M., J. D. Nieland, I. Jochmus, S. Baur, K. Friese, J. Gabelsberger, F. Gieseking, L. Gissmann, B. Glasschroder, T. Grubert, P. Hillemanns, R. Hopfl, H. Ikenberg, J. Schwarz, M. Karrasch, A. Knoll, V. Kuppers, M. Lechmann, R. J. Lelle, H. Meissner, R. T. Muller, M. Pawlita, K. U. Petry, H. Pilch, E. Walek, and A. Schneider. 2007. Vaccination trial with HPV 16 L1 E7 chimeric virus-like particles in women suffering from high grade cervical intraepithelial neoplasia (CIN 2/3). International journal of cancer. Journal international du cancer 121:2794-2800.
50. Carbone, F. R., and M. J. Bevan. 1990. Class I-restricted processing and presentation of exogenous cell-associated antigen in vivo. The Journal of experimental medicine 171:377-387.
51. Hallez, S., J. M. Brulet, C. Vandooren, F. Maudoux, S. Thomas, M. Heinderickx, A. Bollen, R. Wattiez, and A. Jacquet. 2004. Pre-clinical immunogenicity and anti-tumour efficacy of a deleted recombinant human papillomavirus type 16 E7 protein. Anticancer Research 24:2265-2275.
52. Welters, M. J., D. V. Filippov, S. J. van den Eeden, K. L. Franken, J. Nouta, A. R. Valentijn, G. A. van der Marel, H. S. Overkleeft, G. Lipford, R. Offring a, C. J. Melief, J. H. van Boom, S. H. van der Burg, and J. W. Drijfhout. 2004. Chemically synthesized protein as tumour-specific vaccine: immunogenicity and efficacy of synthetic HPV16 E7 in the TC-1 mouse tumour model. Vaccine 23:305-311.
53. Tobian, A. A., D. H. Canaday, W. H. Boom, and C. V. Harding. 2004. Bacterial heat shock proteins promote CD91-dependent class I MHC cross-presentation of chaperoned peptide to CD8+ T cells by cytosolic mechanisms in dendritic cells versus vacuolar mechanisms in macrophages. Journal of immunology (Baltimore, Md.: 1950) 172:5277-5286.
54. Tobian, A. A., C. V. Harding, and D. H. Canaday. 2005. Mycobacterium tuberculosis heat shock fusion protein enhances class I MHC cross-processing and -presentation by B lymphocytes. Journal of immunology (Baltimore, Md.: 1950) 174:5209-5214.
55. Salio, M., and V. Cerundolo. 2005. Viral immunity: cross-priming with the help of TLR3. Current biology: CB 15:R336-339.
56. Zaks, K., M. Jordan, A. Guth, K. Sellins, R. Kedl, A. Izzo, C. Bosio, and S. Dow. 2006. Efficient immunization and cross-priming by vaccine adjuvants containing TLR3 or TLR9 agonists complexed to cationic liposomes. Journal of immunology (Baltimore, Md.: 1950) 176:7335-7345.
57. Heng, B. et al. Human papilloma virus is associated with breast cancer. Br J Cancer (2009).
58. Frazer, I. H. Prevention of cervical cancer through papillomavirus vaccination. Nature reviews. Immunology 4, 46-54 (2004).
59. Collins, S. et al. High incidence of cervical human papillomavirus infection in women during their first sexual relationship. Bjog 109, 96-98 (2002).
60. Cutts, F. T. et al. Human papillomavirus and HPV vaccines: a review. Bull World Health Organ 85, 719-726 (2007).
61. Leggatt, G. R. & Frazer, I. H. HPV vaccines: the beginning of the end for cervical cancer. Curr Opin Immunol 19, 232-238 (2007).
62. Moscicki, A. B. HPV Vaccines: today and in the Future. J Adolesc Health 43, S26-40 (2008).
63. Monie, A., Tsen, S. W., Hung, C. F. & Wu, T. C. Therapeutic HPV DNA vaccines. Expert Rev Vaccines 8, 1221-1235 (2009).
64. Bermudez-Humaran, L. G. & Langella, P. Perspectives for the development of human papillomavirus vaccines and immunotherapy. Expert Rev Vaccines 9, 35-44 (2010).
65. Cid-Arregui, A. Therapeutic vaccines against human papillomavirus and cervical cancer. Open Virol J 3, 67-83 (2009).
66. Zinkemagel, R. M. & Hengartner, H. Protective ‘immunity’ by pre-existent neutralizing antibody titers and preactivated T cells but not by so-called ‘immunological memory’. Immunol Rev 211, 310-319 (2006).
67. Pantaleo, G. & Koup, R. A. Correlates of immune protection in HIV-1 infection: what we know, what we don't know, what we should know. Nat Med 10, 806-810 (2004).
68. Peng, S. et al. Cluster intradermal DNA vaccination rapidly induces E7-specific CD8+ T-cell immune responses leading to therapeutic antitumor effects. Gene Ther 15, 1156-1166 (2008).
69. Murali-Krishna, K. et al. Counting antigen-specific CD8 T cells: a reevaluation of bystander activation during viral infection. Immunity 8, 177-187 (1998).
70. Lu, S. Heterologous prime-boost vaccination. Curr Opin Immunol 21, 346-351 (2009).
71. Radosevic, K., Rodriguez, A., Lemckert, A. & Goudsmit, J. Heterologous prime-boost vaccinations for poverty-related diseases: advantages and future prospects. Expert Rev Vaccines 8, 577-592 (2009).
72. Ranasinghe, C. & Ramshaw, I. A. Genetic heterologous prime-boost vaccination strategies for improved systemic and mucosal immunity. Expert Rev Vaccines 8, 1171-1181 (2009).
73. Altschul, S. F. 1991. “Amino acid substitution matrices from an information theoretic perspective.” Journal of Molecular Biology, 219: 555-665.
74. Dayhoff, M. O., Schwartz, R. M., Orcutt, B. C. 1978. “A model of evolutionary change in proteins.” In “Atlas of Protein Sequence and Structure” 5(3) M. O. Dayhoff (ed.), 345-352, National Biomedical Research Foundation, Washington;
75. States, D. J., Gish, W., Altschul, S. F. 1991. “Improved Sensitivity of Nucleic Acid Database Search Using Application-Specific Scoring Matrices” Methods: A companion to Methods in Enzymology 3(1): 66-77;
76. Steven Henikoff and Jorja G. Henikoff. 1992 “Amino acid substitution matrices from protein blocks.” Proc. Natl. Acad. Sci. USA. 89(biochemistry): 10915-10919;
77. M. S. Johnson and J. P. Overington. 1993. “A Structural Basis of Sequence Comparisons: An evaluation of scoring methodologies.” Journal of Molecular Biology. 233: 716-738.
78. Steven Henikoff and Jorja G. Henikoff. 1993. “Performance Evaluation of Amino Acid Substitution Matrices.” Proteins: Structure, Function, and Genetics. 17: 49-61;
79. Karlin, S, and Altschul, S. F. 1990. “Methods for assessing the statistical significance of molecular sequence features by using general scoring schemes” Proc. Natl. Acad. Sci. USA. 87: 2264-2268.)
80. Eisenberg et al (J. Mol. Bio. 179:125-142, 184.
81. Sambrook, et al. Molecular Cloning: A Laboratory Manual. 2^{nd} ed., Cold Spring Harbor Laboratory, Cold Spring Harbor Laboratory Press, Cold Spring Harbor, N.Y., 1989.
82. Ausubel et al. Current Protocols in Molecular Biology, John Wiley & Sons, 1994).
83. Remington's Pharmaceutical Sciences” (19^{th} edition), ed. A. Gennaro, 1995, Mack Publishing Company, Easton, Pa.

1. A polypeptide comprising an amino acid sequence substantially identical to the amino acid sequence of two or more human papillomavirus (HPV) E7 antigens, wherein the E7 antigens are selected from at least two different HPV strains.

2. The polypeptide of claim 1, consisting essentially of an amino acid sequence substantially identical to the amino acid sequence of two or more human papillomavirus (HPV) E7 antigens, wherein the E7 antigens are selected from at least two different HPV strains.

3. The polypeptide of claim 1, wherein the HPV strains are selected from two or more of the group consisting of HPV16, HPV18, HPV31, HPV33, HPV35, HPV39, HPV45, HPV51, HPV52, HPV56, HPV58, HPV59, HPV68, HPV73, and HPV82.

4.-7. (canceled)

8. The polypeptide of claim 1, wherein the polypeptide comprises the amino acid sequence set forth in SEQ ID NO: 16 or 17.

9.-19. (canceled)

20. A nucleic acid molecule encoding the polypeptide of claim 1.

21.-22. (canceled)

23. A composition comprising the polypeptide of claim 1 and further comprising a carrier.

24. (canceled)

25. The composition of claim 23 further comprising an adjuvant.

26. The composition of claim 25 wherein the adjuvant is selected from the group consisting of a Toll-like receptor (TLR) agonist, an interferon-alpha, an agonist of the 4-1 BB receptor, an agonist of the CD40 receptor, or an anti-CD40 antibody.

27. The composition of claim 26 wherein the TLR agonist is a TLR3 agonist or a TLR9 agonist.

28. The composition of claim 27 wherein the TLR3 agonist is poly(I:C).

29. The composition of claim 27 wherein the TLR9 agonist is a CpG containing oligonucleotide.

30. (canceled)

31. A method of stimulating an immune response, or of treating or preventing a condition associated with HPV infection, or of treating a HPV infection, in a subject in need thereof, the method comprising administering an effective amount of the polypeptide of claim 1 to the subject.

32. (canceled)

33. The method of claim 31 wherein the condition associated with HPV infection is selected from the group consisting of one or more of a cancer of the breast, cervix, anus, vulva, vagina, penis, head and neck, and lung, or pre-malignant lesion thereof.

34. The method of claim 31 wherein the condition associated with HPV infection is a pre-cancerous cervical epithelial neoplasia (CIN I through CIN III) or a cervical cancer.

35.-36. (canceled)

37. The method of any one of claims 31, 33, or 34 wherein the high risk HPV type is selected from one or more of the group consisting of HPV16, HPV18, HPV31, HPV33, HPV35, HPV39, HPV45, HPV51, HPV52, HPV56, HPV58, HPV59, HPV68, HPV73, and HPV82.

38. The method of any one of claims 31, 33, 34, or 37 further comprising administering an adjuvant.

39. The method of claim 38 wherein the adjuvant is selected from the group consisting of a Toll-like receptor (TLR) agonist, an interferon-alpha, an agonist of the 4-1 BB receptor, an agonist of the CD40 receptor, or an anti-CD40 antibody.

40.-43. (canceled)

44. The method of any one of claims 31, 33, 34, or 37-39 wherein the administering comprises administration of multiple doses over a time frame of less than 14 days or comprises administration of multiple doses over one to four days or comprises administration of multiple daily doses.

45.-51. (canceled)

52. A peptide consisting essentially of one or more of the amino acid sequences TSNYNIVTF (SEQ ID NO: 35), AEPDTSNYNIVTFCC (SEQ ID NO: 36) or TSNYNIVTFCCQCKS (SEQ ID NO: 37).

53. A method of diagnosing a HPV31 infection or of determining the response of a subject to a HPV31 infection, the method comprising contacting a sample with a peptide consisting essentially of one or more of the amino acid sequences TSNYNIVTF (SEQ ID NO: 35), AEPDTSNYNIVTFCC (SEQ ID NO: 36) or TSNYNIVTFCCQCKS (SEQ ID NO: 37).

54. (canceled)
